SEQUENCE LISTING (1 sequence)

SEQ ID NO: 1 - protein, 210 aa, Homo sapiens
MADGSSDAAREPRPAPAPIRRRSSNYRAYATEPHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

(19) United States
(12) Patent Application Publication
(10) Pub. No.: US 2026/0348978 A1
(43) Pub. Date: Nov. 5, 2026

(54) NOVEL BIOMARKER OF ACUTE CORONARY SYNDROMES

(71) Applicant: OTAGO INNOVATION LIMITED, Dunedin (NZ)

(72) Inventors: Sara Dairne Raudsepp, Dunedin (NZ); Christopher Pemberton, Christchurch (NZ)

(21) Appl. No.: 19/116,457

(22) PCT Filed: Sep. 9, 2023
(86) PCT No.: PCT/NZ2023/050104

§ 371 Date: Mar. 28, 2025

(30) Foreign Application Priority Data
Sep. 30, 2022 (AU) 2022902829

(51) Int. Cl. G01N 33/68; G01N 33/58

(52) U.S. Cl. CPC G01N 33/6887; G01N 33/581; G01N 2333/4712; G01N 2800/324

(57) ABSTRACT

The present invention is concerned with a novel approach to the measurement of circulating biomarkers of cardiac disease. In particular, the present invention provides assays, methods and test kits for the detection of unbound circulating cardiac troponins, such as unbound cardiac troponin I (cTnI), and to the utility of these biomarkers for diagnosing cardiac disease in a patient (e.g. acute coronary syndromes including acute myocardial infarction and unstable angina pectoris) or for predicting future stroke or mortality. The present invention further provides a unique approach to enhance the clinical performance/accuracy of existing troponin assays in triaging patients with an acute myocardial infarction for immediate therapeutic interventions again by measuring unbound cardiac troponins, such as cTnI.

### TECHNICAL FIELD

The present invention is concerned with a novel approach to the measurement of circulating biomarkers of cardiac disease. In particular, the present invention provides assays, methods and test kits for the detection of unbound circulating cardiac troponins and to the utility of these biomarkers for identifying cardiac disease in a patient, for example, acute coronary syndromes such as acute myocardial infarction and unstable angina pectoris.

### BACKGROUND OF THE INVENTION

The following includes information that may be useful in understanding the present invention. It is not an admission that any of the information, publications or documents specifically or implicitly referenced herein is prior art, or essential, to the presently described or claimed inventions. All publications and patents mentioned herein are hereby incorporated herein by reference in their entirety.

Approximately 65,000 patients present annually to hospital with chest pain in New Zealand making it one of the most common causes for presentation [1]. In these patients, the accurate and timely diagnosis of acute coronary syndromes (ACS, comprising myocardial infarction and unstable angina) is of major importance due to the high prevalence (~25% of ACS patients), mortality and morbidity (6-fold increased risk of major adverse event within 2 years) associated with these conditions [2, 7]. The diagnosis of myocardial infarction is made on the basis of thorough clinical evaluation and measurement of circulating cardiac troponins (cTn). More recently, the introduction of highly sensitive (hs) troponin assays has facilitated faster assessment pathways for myocardial infarction diagnosis [3-5]. However, a limitation with existing troponin assays is the inability to take account of troponin-troponin binding interactions (e.g.) a binding complex comprising cardiac troponin T and cardiac troponin I (cTnI-cTnT). Further, the inability to distinguish between physiological levels of troponin and troponin produced by infarcted myocardial tissue is also a limitation associated with existing commercial troponin assays. Accordingly, improvements in the specificity of universally adopted clinical tests such as (e.g.) Roche's high sensitivity troponin assay (hsTnT) would yield improved patient triage and treatment outcomes for the management of acute myocardial infarction.

By comparison, early diagnosis of unstable angina (i.e. a different acute coronary syndrome) from clinical signs, symptoms, and blood tests is more difficult, often requiring time consuming invasive or provocative procedures such as angiography and stress testing [3]. As yet, no circulating biomarkers provide clinically useful information to aid the rapid diagnosis of unstable angina from other confounding diagnoses such as aortic dissection, pericarditis or pulmonary embolism and musculoskeletal chest pain [6]. Accordingly, there is a major unmet clinical need for markers that could rapidly distinguish unstable angina from other non-cardiac causes of chest pain, a problem that has major significance given that unstable angina is an important precursor for future myocardial infarction and has its own significant morbidity [7].

In its recent clinical evaluation and assay development work, the Applicant has made a surprising discovery which addresses these and other unmet clinical needs as contemplated by the data and inventions described herein.

### SUMMARY OF THE INVENTION

The inventions described and claimed herein have many attributes and embodiments including, but not limited to, those set forth or described or referenced in this Summary of the Invention. It is not intended to be all inclusive and the inventions described and claimed herein are not limited to or by the features or embodiments identified in this Summary of the Invention, which is included for purposes of illustration only and not restriction.

In an aspect of the present invention there is provided a test kit for determining the level of a bound protein complex comprising cardiac Troponin I (cTnI) and an immunoglobulin G (IgG) in a biological sample obtained from a patient, the test kit comprising:
(i) an anti-cTnI antibody which selectively binds to an equivalent region comprising amino acids 80-97 of cTnI as defined by SEQ ID NO: 1; and
(ii) an anti-IgG antibody which selectively binds to the IgG which is bound to cTnI.

In another aspect of the present invention there is provided a method for determining the level of a bound protein complex comprising cardiac Troponin I (cTnI) and an immunoglobulin G (IgG) (bTnI-IgG) in a biological sample obtained from a patient, the method comprising:
(i) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
(ii) washing the reaction mix from (i) to remove non-selectively bound analytes;
(iii) contacting the reaction mix from (ii) with an anti-IgG detection antibody for a time and under conditions sufficient for the anti-IgG detection antibody to selectively bind the bTnI-IgG complex; and
(iv) determining the level of bTnI-IgG in the biological sample.

In a further aspect of the present invention there is provided a method for diagnosing an acute coronary syndrome in a patient, the method comprising:
(i) determining the level of a bound protein complex comprising cardiac Troponin I (cTnI) and an immunoglobulin G (IgG) (bTnI-IgG) in a biological sample obtained from the patient which comprises:
   (a) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
   (b) washing the reaction mix from (a) to remove non-selectively bound analytes;
   (c) contacting the reaction mix from (b) with an anti-IgG detection antibody, for a time and under conditions sufficient for the anti-IgG detection antibody to selectively bind to the bTnI-IgG complex; and
   (d) determining the level of bTnI-IgG in the biological sample;
(ii) comparing the level of bTnI-IgG from (i) against a reference standard from a control population
wherein, a lower level of bTnI-IgG in the biological sample measured relative to the reference standard from a control population is indicative that the patient has an acute coronary syndrome.

In a further aspect of the present invention there is provided a method for diagnosing unstable angina pectoris in a patient, the method comprising:
(i) determining the level of a bound protein complex comprising cardiac Troponin I (cTnI) and an immunoglobulin G (IgG) (bTnI-IgG) in a biological sample obtained from the patient which comprises:
   (a) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
   (b) washing the reaction mix from (a) to remove non-selectively bound analytes;
   (c) contacting the reaction mix from (b) with an anti-IgG detection antibody, for a time and under conditions sufficient for the anti-IgG detection antibody to selectively bind to the bTnI-IgG complex; and
   (d) determining the level of bTnI-IgG in the biological sample;
(ii) comparing the level of bTnI-IgG from (i) against a reference standard from a control population
wherein, a lower level of bTnI-IgG in the biological sample measured relative to the reference standard from a control population is indicative that the patient has unstable angina pectoris.

In yet another aspect of the present invention there is provided a method for enhancing the diagnostic performance of a cardiac troponin T (cTnT) assay for the diagnosis of an acute myocardial infarction in a patient, the method comprising:
(i) determining the level of a bound protein complex comprising cardiac Troponin I (cTnI) and an immunoglobulin G (IgG) (bTnI-IgG) in a biological sample obtained from the patient which comprises:
   (a) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
   (b) washing the reaction mix from (a) to remove non-selectively bound analytes;
   (c) contacting the reaction mix from (b) with an anti-IgG detection antibody, for a time and under conditions sufficient for the anti-IgG detection antibody to selectively bind to the bTnI-IgG complex; and
   (d) determining the level of bTnI-IgG in the biological sample;
(ii) comparing the level of bTnI-IgG from (i) against a reference standard from a control population
wherein, when the level of bTnI-IgG in the biological sample is lower compared to the level of bTnI-IgG of a reference standard from a control population it is combined with cTnT to improve the diagnostic accuracy of cTnT which is achieved in the absence of bTnI-IgG.

In yet a further aspect of the present invention there is provided a method for predicting a future acute coronary syndrome event in a patient, the method comprising:
(i) determining the level of a bound protein complex comprising cardiac Troponin I (cTnI) and an immunoglobulin G (IgG) (bTnI-IgG) in a biological sample obtained from the patient which comprises:
   (a) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
   (b) washing the reaction mix from (a) to remove non-selectively bound analytes;
   (c) contacting the reaction mix from (b) with an anti-IgG detection antibody, for a time and under conditions sufficient for the anti-IgG detection antibody to selectively bind to the bTnI-IgG complex; and
   (d) determining the level of bTnI-IgG in the biological sample;
(ii) comparing the level of bTnI-IgG from (i) against a reference standard from a control population
wherein, a lower level of bTnI-IgG in the biological sample measured relative to the reference standard from a control population is predictive of a future acute coronary syndrome event in the absence of a therapeutic intervention.

In yet a further aspect of the present invention there is provided a method for predicting stroke in a patient, the method comprising:
(i) determining the level of a bound protein complex comprising cardiac Troponin I (cTnI) and an immunoglobulin G (IgG) (bTnI-IgG) in a biological sample obtained from the patient which comprises:
   (a) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
   (b) washing the reaction mix from (a) to remove non-selectively bound analytes;
   (c) contacting the reaction mix from (b) with an anti-IgG antibody, for a time and under conditions sufficient for the anti-IgG antibody to selectively bind to the bTnI-IgG complex; and
   (d) determining the level of bTnI-IgG in the biological sample;
(ii) comparing the level of bTnI-IgG from (i) against a reference standard from a control population
wherein, a lower level of bTnI-IgG in the biological sample measured relative to the reference standard from a control population is predictive that the patient could suffer from a future stroke in the absence of a therapeutic intervention.

In yet a further aspect of the present invention there is provided a method for predicting mortality in a patient, the method comprising:
(i) determining the level of a bound protein complex comprising cardiac Troponin I (cTnI) and an immunoglobulin G (IgG) (bTnI-IgG) in a biological sample obtained from the patient which comprises:
   (a) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
   (b) washing the reaction mix from (a) to remove non-selectively bound analytes;
   (c) contacting the reaction mix from (b) with an anti-IgG detection antibody, for a time and under conditions sufficient for the anti-IgG detection antibody to selectively bind to the bTnI-IgG complex; and
   (d) determining the level of bTnI-IgG in the biological sample;
(ii) comparing the level of bTnI-IgG from (i) against a reference standard from a control population
wherein, a lower level of bTnI-IgG in the biological sample measured relative to the reference standard from a control population is predictive of future mortality in the absence of a therapeutic intervention.

In a further aspect of the present invention there is provided a peptide complex comprising cardiac troponin I (cTnI) and a human immunoglobulin G (IgG) which is bound to a binding agent which selectively binds to cTnI, and preferably to an equivalent region defined by amino acid residues 80-97, more preferably amino acid residues 86-90, of cTnI as defined by SEQ ID NO: 1.

In another further aspect of the present invention there is provided a peptide complex comprising cardiac troponin I (cTnI) and a human immunoglobulin G (IgG) which is bound to a monoclonal antibody or antigen-binding fragment thereof which selectively binds to cTnI, and preferably to an equivalent region defined by amino acid residues 80-97, more preferably amino acid residues 86-90, of cTnI as defined by SEQ ID NO: 1.

In yet another aspect of the present invention there is provided a peptide complex comprising cardiac troponin I (cTnI) and a human immunoglobulin G (IgG) which is bound to an aptamer which selectively binds to cTnI, and preferably to an equivalent region defined by amino acid residues 80-97, more preferably amino acid residues 86-90, of cTnI as defined by SEQ ID NO: 1.

In yet a further aspect of the present invention there is provided a binding agent comprising a peptide framework comprising one or more complementarity determining regions derived from an antibody which selectively binds to cardiac troponin I (cTnI) or cardiac troponin T (cTnT).

In yet a further aspect of the present invention there is provided a binding agent comprising a peptide framework comprising three complementarity determining regions derived from an antibody which selectively binds to cardiac troponin I (cTnI) or cardiac troponin T (cTnT).

In yet another aspect of the present invention there is provided an aptamer or aptamer ligand binding domain which selectively binds to cardiac troponin I (cTnI) or cardiac troponin T (cTnT).

In yet a further aspect of the present invention there is provided a binding agent which selectively binds to cardiac troponin I (cTnI) or cardiac troponin T (cTnT).

In yet another aspect of the present invention there is provided an antibody or antigen-binding fragment which selectively binds to cardiac troponin I (cTnI) or cardiac troponin T (cTnT).

In yet a further aspect of the present invention there is provided a monoclonal antibody, a polyclonal antibody, a chimeric antibody or a humanized antibody which selectively binds to IGFBP-3, or an antigen-binding fragment of a monoclonal, polyclonal, chimeric or humanized antibody which selectively binds to cardiac troponin I (cTnI) or cardiac troponin T (cTnT).

In a further aspect of the present invention there is provided a method for diagnosing an acute coronary syndrome in a patient, the method comprising:
(i) determining the level of a bound protein complex comprising cardiac Troponin I (cTnI) and an immunoglobulin G (IgG) (bTnI-IgG) in a biological sample obtained from the patient which comprises:
   (a) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
   (b) washing the reaction mix from (a) to remove non-selectively bound analytes;
   (c) contacting the reaction mix from (b) with an anti-IgG detection antibody, for a time and under conditions sufficient for the anti-IgG detection antibody to selectively bind to the bTnI-IgG complex; and
   (d) determining the level of bTnI-IgG in the biological sample;
(ii) comparing the level of bTnI-IgG from (i) against a reference standard from a control population wherein a lower level of bTnI-IgG in the biological sample compared to the reference standard from the control population is indicative that the patient has an acute coronary syndrome; and
(iii) where the level of bTnI-IgG in the biological sample is lower than the reference standard from the control population, triaging and/or treating the human for the acute coronary syndrome.

In another aspect of the present invention there is provided a method for determining the level of a bound protein complex comprising cardiac Troponin I (cTnI) and an immunoglobulin G (IgG) (bTnI-IgG) in a biological sample obtained from a patient, the method comprising:
(i) contacting the biological sample with a reaction mix comprising an anti-cTnI capture binding member which selectively binds to an equivalent region comprising amino acids 80-97 of cTnI as defined by SEQ ID NO: 1, for a time and under conditions sufficient for the anti-cTnI capture binding member to selectively bind to the bTnI-IgG complex;
(ii) washing the reaction mix from (i) to remove non-selectively bound analytes;
(iii) contacting the reaction mix from (ii) with an anti-IgG detection binding member, for a time and under conditions sufficient for the anti-IgG detection binding member to selectively bind to the bTnI-IgG complex; and
(iv) determining the level of bTnI-IgG in the biological sample.

In another aspect of the present invention there is provided a method for determining the level of a bound protein complex comprising cardiac Troponin I (cTnI) and an immunoglobulin G (IgG) (bTnI-IgG) in a biological sample obtained from a patient, the method comprising:
(i) contacting the biological sample with a reaction mix comprising an anti-cTnI capture aptamer which selectively binds to an equivalent region comprising amino acids 80-97 of cTnI as defined by SEQ ID NO: 1, for a time and under conditions sufficient for the anti-cTnI capture aptamer to selectively bind to the bTnI-IgG complex;
(ii) washing the reaction mix from (i) to remove non-selectively bound analytes;
(iii) contacting the reaction mix from (ii) with an anti-IgG detection aptamer, for a time and under conditions sufficient for the anti-IgG detection aptamer to selectively bind to the bTnI-IgG complex; and
(iv) determining the level of bTnI-IgG in the biological sample.

### BRIEF DESCRIPTION OF THE FIGURES

FIG. 1A shows a schematic illustrating the principles of an bTnI-IgG assay according to the present invention. Importantly, the assay depicted in this schematic will not bind a troponin complex comprising cardiac troponin T and cardiac troponin I (i.e. cTnT-cTnI). Only cardiac troponin I that is not complexed with cardiac troponin T will bind to an anti-TnI antibody because the anti-TnI antibody binds to exactly the same region/epitope of TnI to which TnT binds. The amount of “free” or “unbound” TnI is then measured using an anti-IgG detection antibody which targets the IgG bound to TnI.

FIG. 2 shows mean bTnI-IgG median inter-quartile range absorbance in 1053 chest pain patients with an adjudicated diagnosis of NCCP (n=427), possible UAP (n=291), other cardiac disorders (n=46), definitive UAP (n=72), NSTEMI (n=182) and STEMI (n=35).

FIG. 3A shows Receiver Operating Curve (ROC) performance for (i) hsTnI (AUC=0.954; blue line), (ii) hsTnT (AUC=0.925; green line) and (iii) bTnI-IgG (AUC=0.442; orange line) for patients with an adjudicated diagnosis of myocardial infarction (n=217) from within the SPACE cohort. In these measurements all concentrations of bTnI-IgG were used in the ROC analyses; the median of bTnI-IgG was 0.9659 U.

FIG. 3B shows Receiver Operating Curve (ROC) performance for (i) hsTnI (AUC=0.955; blue line), (ii) hsTnT (AUC=0.958; green line) and (iii) bTnI-IgG (AUC=0.362; orange line) for patients with an adjudicated diagnosis of myocardial infarction from within the SPACE cohort. In these measurements only bTnI-IgG above the median (i.e. >0.9659 U) was taken into account for the ROC analyses.

FIG. 3C shows Receiver Operating Curve (ROC) performance for (i) hsTnI (AUC=0.952; blue line), (ii) hsTnT (AUC=0.908; green line) and (iii) bTnI-IgG (AUC=0.584; orange line) for patients with an adjudicated diagnosis of myocardial infarction from within the SPACE cohort. In these measurements only bTnI-IgG below the median (i.e. <0.9659 U) was taken into account in the ROC analyses.

FIG. 4 shows Receiver Operating Curve (ROC) performance for (i) hsTnT (AUC=0.76; green line), (ii) hsTnT+bTnI-IgG (AUC=0.81; blue line) and (iii) bTnI-IgG (AUC=no data/no statistical relevance; orange line) where hsTnT is less than or equal to 15 ng/L at index presentation and is used to diagnose myocardial infarction.

FIG. 5 shows Receiver Operating Curve (ROC) performance for (i) hsTnT (AUC=0.60; green line), (ii) hsTnT+bTnI-IgG (AUC=0.67; blue line) where hsTnT is less than or equal to 15 ng/L at index presentation and is used to rule out myocardial infarction.

FIG. 6A shows Receiver Operating Curve (ROC) performance for (i) hsTnI (AUC=0.955±0.008; green line), (ii) hsTnI+bTnI-IgG (AUC=0.955±0.008; blue line) and (iii) bTnI-IgG (AUC=0.441±0.020; orange line) for patients with an adjudicated diagnosis of myocardial infarction (n=217) from within the SPACE cohort. In these measurements all concentrations of hsTnI were used in the ROC analyses.

FIG. 6B shows Receiver Operating Curve (ROC) performance for (i) hsTnI (AUC=0.888±0.020; green line), (ii) hsTnI+bTnI-IgG (AUC=0.889±0.020; blue line) and (iii) bTnI-IgG (AUC=0.422±0.034; orange line) for patients with an adjudicated diagnosis of myocardial infarction (n=217) from within the SPACE cohort. In these measurements only hsTnI concentrations of less than or equal to 30 ng/L were used in the ROC analyses.

FIG. 7 shows Receiver Operating Curve (ROC) performance for (i) calc age @admission (AUC=0.826±0.039; green line), (ii) calc age @admission+bTnI-IgG (AUC=0.850±0.035; blue line) and (iii) bTnI-IgG (AUC=0.289±0.041; orange line) for patients with an adjudicated diagnosis of stroke within 2y of index presentation to the SPACE cohort (n=27/1053).

FIG. 8 shows Receiver Operating Curve (ROC) performance for (i) bTnI-IgG (AUC=0.322±0.030; light green line), (ii) calc age @admission (AUC=0.854±0.020; dark green line), (iii) NT-proBNP (AUC=0.866±0.018; light brown line), (iv) entire model (AUC=0.896±0.017; orange line), and (v) calc age @admission+bTnI-IgG (AUC=0.857±0.019; blue line) for patients who reached mortality within 4 years of index presentation to the SPACE cohort (n=90/1053).

FIG. 9 shows the human cardiac troponin I amino acid sequence (i.e. a total of 210 amino acid residues). The region defined by amino acid residues 80-97 is represented in bold, and within that the region defined by amino acids 86-90 is additionally underlined.

FIG. 10 shows Receiver Operating Curve (ROC) performance for (i) hsTnT (AUC=0.920; green line) and (ii) hsTnT+bTnI-IgG (AUC=0.922; blue line) and (iii) hsTnT+bTnI-IgG+sex+abnormal electrocardiogram (AUC=0.929; red line) for patients with an adjudicated diagnosis of myocardial infarction (n=678) derived from a combined patient cohort comprising the SPACE, FAST-TRAC and APACE (n=4276).

FIG. 11A shows Receiver Operating Curve (ROC) performance for (i) hsTnT (AUC=0.757; red line), (ii) hsTnT+bTnI-IgG (AUC=0.783; blue line) and (iii) bTnI-IgG (AUC=0.379; green line) for patients with an adjudicated diagnosis of myocardial infarction (n=54/2809) when hsTnT at index presentation is <14 ng/L.

FIG. 11B shows Receiver Operating Curve (ROC) performance for (i) hsTnT+abnormal ECG (AUC=0.801; red line), (ii) hsTnT+bTnI-IgG (AUC=0.783; blue line) and (iii) hsTnT+abnormal ECG+bTnI-IgG (AUC=0.815; green line) for patients with an adjudicated diagnosis of myocardial infarction (n=54/2809) when hsTnT at index presentation is <14 ng/L.

FIG. 12 shows a schematic depicting a generalised linear regression model for (A) prediction of all myocardial infarction (B) prediction of Type 1 myocardial infarction and (C) prediction of Type 2 myocardial infarction where hsTnT at index presentation is >14 ng/L. The total number of patient samples interrogated was 1458 of which 633 had a myocardial infarction (542 Type 1 MI and 91 Type 2 MI). Note, “Marker D”=bTnI-IgG.

FIG. 13 shows Receiver Operating Curve (ROC) performance for (i) heart rate (AUC=0.596±0.034; blue line), (ii) heart rate+bTnI-IgG (AUC=0.671±0.034; red line), (iii) hsTnT+bTnI-IgG (AUC=0.581±0.027; turquoise line), (iv) hsTnT (AUC=0.556±0.029; purple line) and bTnI-IgG (AUC=0.369±0.028; green line) to identify patients with Type 2 myocardial infarction (n=94/1494 tested) where hsTnT at index presentation >14 ng/L.

FIG. 14A shows Receiver Operating Curve (ROC) performance for (i) hsTnT0 (AUC=0.886±0.007; green line), (ii) DBP (AUC=0.550±0.014; light blue line), (iii) heart rate (AUC=0.485±0.013; red line), (iv) bTnI-IgG (AUC=0.421±0.013; dark blue line), (v) heart rate+bTnI-IgG (AUC=0.548±0.013; turquoise line) and (vi) heart rate+bTnI-IgG+DBP (AUC=0.541±0.013; purple line) to identify patients with Type 1 myocardial infarction (n=579/3026 tested; 98.4% of all Type 1 MI) where hsTnT at index presentation>5 ng/L.

FIG. 14B shows Receiver Operating Curve (ROC) performance for (i) hsTnT0 (AUC=0.755±0.017; green line), (ii) DBP (AUC=0.455±0.030; light blue line), (iii) heart rate (AUC=0.608±0.032; red line), (iv) bTnI-IgG (AUC=0.328±0.026; dark blue line), (v) heart rate+bTnI-IgG (AUC=0.704±0.027; turquoise line) and (vi) heart rate+bTnI-IgG+DBP (AUC=0.721±0.027; purple line) to identify patients with Type 2 myocardial infarction (n=103/3026 tested; 100% of all Type 2 MI) where hsTnT at index presentation>5 ng/L.

FIG. 15 shows 99^{th} percentile estimates of heart rate+bTnI-IgG in myocardial infarction.

FIG. 16 shows Receiver Operating Curve (ROC) performance for (i) AbTnI-IgG (AUC=0.646±0.043; blue line), (ii) hsTnT (AUC=0.488±0.042; red line), (iii) heart rate+DPB+platelet count+hemaglobin levels (AUC=0.711±0.050; purple line) and (iv) heart rate+DPB+hemaglobin levels+deltaMD (AUC=0.793±0.035; turquoise line) to identify patients with Type 2 myocardial infarction (n=94/1494 tested) where hsTnT at index presentation>14 ng/L.

FIG. 17 shows a clinical stratification chart for (A) presentation troponin only and (B) troponin levels between 0-2 h.

FIG. 18 shows the utility of bTnI-IgG to predict a new acute coronary syndrome within 1 year (AUC=0.832±0.020; red line), optionally in conjunction with IGFBP3 (AUC=0.835±0.020; red line), in a patient with no diagnosis of myocardial infarction at index presentation.

FIG. 19 shows the utility of bTnI-IgG to predict a new acute coronary syndrome within 1 year in patients with a significant history of cardiovascular disease including (e.g.) Type 2 myocardial infarction, unstable angina pectoris or radiology findings of 70% artery occlusion (AUC=0.664±0.030; blue line).

FIG. 20 shows the utility of bTnI-IgG to predict a new acute coronary syndrome within one year in patients who present with a positive angiogram, positive stress test or a diagnosis of unstable angina pectoris and who had no index diagnosis of myocardial infarction (41/333; AUC=0.718; turquoise line).

FIG. 21 shows the utility of bTnI-IgG to predict a positive stress test from within the BASEL VIII cohort (n=21/49).

### DETAILED DESCRIPTION

### General Definitions

Unless specifically defined otherwise, all technical and scientific terms used herein shall be taken to have the same meaning as commonly understood by one of ordinary skill in the art to which the inventions belong (for example, in immunology, immunohistochemistry, protein chemistry, and biochemistry).

Unless otherwise indicated, the recombinant protein and immunological techniques utilized in the present invention are standard procedures well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al., (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

Any example or embodiment described herein shall be taken to apply mutatis mutandis to any other example or embodiment unless specifically stated otherwise.

### SELECTED DEFINITIONS

The term “ACS” as used herein means acute coronary syndrome. Examples of acute coronary syndromes include, but are not limited to, unstable angina or unstable angina pectoris; cardiac ischemia and myocardial ischemia; Type 1 and Type 2 (acute) myocardial infarction including ST-elevation myocardial infarction (STEMI) and non-ST myocardial infarction (NSTEMI); acute cardiac injury; acute cardiac damage resulting from acute drug toxicity, acute cardiomyopathies and cardiac transplant rejection.

The term “angina” as used herein means any form of chest pain whether that chest pain was experienced historically (e.g. “history of angina”) or in an acute setting.

The acronym “AMI” as used herein is intended to mean acute myocardial infarction.

For any avoidance of doubt the term “history of angina” is taken to mean a patient who has had any history of cardiovascular disease or has a history of chest pain complaints. The terms “history of angina”, “history of cardiovascular disease” and “history of chest pain” as used herein are therefore synonymous.

The term “antibody” refers to an immunoglobulin molecule capable of selectively binding to a target, such as a cardiac troponin (e.g. cTnI), by virtue of an antigen binding site contained within at least one variable region. This term includes four chain antibodies (e.g., two light chains and two heavy chains), recombinant or modified antibodies (e.g., chimeric antibodies, humanized antibodies, primatized antibodies, de-immunized antibodies, half antibodies, bispecific antibodies) and single domain antibodies such as domain antibodies and heavy chain only antibodies (e.g., camelid antibodies or cartilaginous fish immunoglobulin new antigen receptors (IgNARs)). An antibody generally comprises constant domains, which can be arranged into a constant region or constant fragment or fragment crystallisable (Fc). Preferred forms of antibodies comprise a four-chain structure as their basic unit. Full-length antibodies comprise two heavy chains (~50-70 kDa) covalently linked and two light chains (~23 kDa each). A light chain generally comprises a variable region and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ~110 amino acids in length) and one or more constant domains at the C-terminus. The constant domain of the light chain (CL which is ~110 amino acids in length) is aligned with and disulfide bonded to the first constant domain of the heavy chain (CH which is −330-440 amino acids in length). The light chain variable region is aligned with the variable region of the heavy chain. The antibody heavy chain can comprise 2 or more additional CH domains (such as, CH2, CH3 and the like) and can comprise a hinge region can be identified between the CH1 and Cm constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (preferably human) antibody. The term “antibody” encompasses not only intact polyclonal or monoclonal antibodies, but also variants, fusion proteins comprising an antibody portion with an antigen binding site, humanised antibodies, human antibodies, chimeric antibodies, primatised antibodies, de-immunised antibodies or veneered antibodies.

The term “antigen-binding fragment” or “antigen-binding antibody fragment” shall be taken to mean any fragment of an antibody that retains the ability to bind to its target antigen. This term includes a Fab fragment, a Fab′ fragment, a F(ab′) fragment, a single chain antibody (SCA or SCAB) amongst others. A “Fab fragment” consists of a monovalent antigen-binding fragment of an antibody molecule, and can be produced by digestion of a whole antibody molecule with the enzyme papain, to yield a fragment consisting of an intact light chain and a portion of a heavy chain. A “Fab′ fragment” of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fv fragment” is a genetically engineered fragment containing the variable region of a light chain and the variable region of a heavy chain expressed as two chains. A “single chain antibody” (SCA) is a genetically engineered single chain molecule containing the variable region of a light chain and the variable region of a heavy chain, linked by a suitable, flexible polypeptide linker.

The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass, as well as fragments of such antibodies, so long as they exhibit the desired biological activity (U.S. Pat. No. 4,816,567; and Morrison et al. (1984) Proc. Natl Acad. Sci USA 81:6851-6855).

The acronym “DBP” as used herein is intended to mean diastolic blood pressure.

The term “humanized antibody” shall be understood to refer to a chimeric molecule, generally prepared using recombinant techniques, having an epitope binding site derived from an immunoglobulin from a non-human species and the remaining immunoglobulin structure of the molecule based upon the structure and/or sequence of a human immunoglobulin. The antigen-binding site preferably comprises the complementarity determining regions (CDRs) from the non-human antibody grafted onto appropriate framework regions in the variable domains of human antibodies and the remaining regions from a human antibody. Epitope binding sites may be wild type or modified by one or more amino acid substitutions. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When non-human antibodies are prepared with respect to a particular epitope, the variable regions can be “reshaped” or “humanized” by grafting CDRs derived from non-human antibody on the FRs present in the human antibody to be modified.

The term “binding agent” as used herein is intended to refer to any molecule that binds a target antigen and includes isoforms thereof, and the term binding agent includes small molecules, antibodies from any species whether polyclonal or monoclonal, antigen-binding fragments such as Fab and Fab₂, humanized antibodies, chimeric antibodies, or antibodies modified in other ways including substitution of amino acids, and/or fusion with other peptides or proteins (e.g. PEG). It also includes receptors or binding proteins from any species or modified forms of them.

As used herein, the term “antigenic variant” refers to polypeptide sequences different from the specifically identified sequences, wherein one or more amino acid residues are deleted, substituted, or added. Substitutions, additions or deletions of 1, 2, 3 or 4 amino acids are specifically contemplated. Variants may be naturally-occurring allelic antigenic variants, or non-naturally occurring antigenic variants. Variants may be from the same or from other species and may encompass homologues, paralogues and orthologues. In certain embodiments, antigenic variants of the polypeptides useful in the invention have biological activities including hormone function or antigenic-binding properties that are the same or similar to those of the parent polypeptides. The term “antigenic variant” with reference to (poly)peptides encompasses all forms of polypeptides as defined herein. The term “antigenic variant” encompasses naturally occurring, as well as recombinant and synthetic produced polypeptides.

The term “AUC” means Area Under the Curve which yields information about the strength of a correlation determined by the Receiver Operating Curve analysis. Typical ROC values where the AUC is greater than or equal to 0.70 yields a statistically significant correlation.

The term “biological sample” as used herein includes biological fluids selected from blood including venous blood and arterial blood, plasma, serum, intertistial fluid, or any other body fluid. The term “biological sample” also includes heart tissue sample. The term “biological sample” and “body fluid sample” as used herein refers to a biological sample or a sample of bodily fluid obtained for the purpose of, for example, diagnosis, prognosis, classification or evaluation of a subject of interest, such as a patient. In certain embodiments, such a sample may be obtained for diagnosing a cardiac disorder, for performing risk stratification of a cardiac disorder, for making a prognosis of a disease course in a patient with a cardiac disorder, for identifying a patient with elevated risk of a cardiac disorder, or combinations thereof. In addition, a person skilled in the art would realise that certain body fluid samples would be more readily analysed following a fractionation or purification procedure, for example, separation of whole blood into serum or plasma components.

The term “comparing” has used herein has an ordinary meaning attached to it and is intended to mean a side-by-side comparison between the measured level of a particular biomarker from (e.g.) a test sample and the measured level of the same biomarker from a control sample, such as that obtained from an individual or a population of individuals. In other examples, the level of a biomarker measured from a test sample is compared to a test sample taken from an identical patient source at an earlier time point(s).

The terms “control population” and “suitable control population” according to the present invention refers to the mean circulating levels of a biomarker from sex- and age-matched subjects for which their cardiac disease or disorder status is known. The control population is used to provide a suitable reference interval by which a measured (e.g.) protein or isoform level is compared.

The term “Dx” as used herein means diagnosis or diagnostic.

The term “effective amount” as used herein refers to the amount of a therapy that is sufficient to result in the prevention of the development, recurrence, or onset of a disease or condition and one or more symptoms thereof, to enhance or improve the prophylactic effect(s) of another therapy, reduce the severity, the duration of disease, ameliorate one or more symptoms of the disease or condition, prevent the advancement of the disease or condition, cause regression of the disease or condition, and/or enhance or improve the therapeutic effect(s) of another therapy.

The term “ELISA” as used herein means enzyme-linked immunosorbent assay.

The term “epitope” includes any antigenic (e.g., a protein) determinant capable of specific binding to an antibody. Epitope determinants usually consist of chemically active surface groupings of molecules such as amino acids or sugar side chains, and usually have specific three-dimensional structural characteristics, as well as specific charge characteristics. An epitope typically includes, for example, at least 3, 5 or 8-10 amino acids. The amino acids may be contiguous, or non-contiguous amino acids juxtaposed by tertiary folding.

Conformational and non-conformational epitopes are distinguished in that the binding to the former but not the latter is lost in the presence of denaturing solvents.

As used in this specification, the term “fragment” or “functional derivative” in relation to a polypeptide is a subsequence of a polypeptide that may be detected using a binding agent. The term may refer to a polypeptide, an aggregate of a polypeptide such as a dimer or multimer, a fusion polypeptide, a polypeptide fragment, a polypeptide variant or derivative thereof.

The term “hsTnT” as used herein means high sensitivity Troponin T, and includes high sensitivity cardiac Troponin T (i.e. hscTnT).

The term “Hx” as used herein means history, for example “Hx CVD” means history of cardiovascular disease.

The terms “HxHF” or “HFHx” as used herein mean a patient who has a history of heart failure.

The terms “HxMI” or “MIHx” as used herein mean a patient who has a history of myocardial infarction.

An “increase” or “decrease” in the level of a particular biomarker (e.g. bTnI-IgG) compared with a control, or a “change” or “deviation” from a control (level) in one example is statistically significant. An increased level, decreased level, deviation from, or change from a control level or mean or historical control level can be considered to exist if the level differs from the control level by about 5% or more, by about 10% or more, by about 20% or more, or by about 50% or more compared to the control level. Statistically significance may alternatively be calculated as P≤0.05. Increased levels, decreased levels, deviation, and changes can also be determined by recourse to assay reference limits or reference intervals. These can be calculated from intuitive assessment or non-parametric methods. Overall, these methods may calculate the 0.025, and 0.975 fractiles as 0.025*(n+1) and 0.975 (n+1). Such methods are well known in the art. Presence of a marker absent in a control may be seen as a higher level, deviation or change. Absence of a marker present in a control may be seen as a lower level, deviation or change.

The term “index presentation” as used herein means the point at which a patient presents to (e.g.) an emergency department, a clinic, a hospital, a surgery, a doctor's practice, a doctor or any other relevant medical forum, and information about the cardiac status of the patient is measured, including the patient's cardiac troponin levels levels. For any avoidance of doubt, the term “index presentation” also includes determining the levels of cardiac troponin(s) in a patient or subject who has a new or recurring complaint of chest pain.

The term “isolated” as applied to the polypeptide sequences disclosed herein is used to refer to sequences that are removed from their natural cellular or other naturally-occurring biological environment. An isolated molecule may be obtained by any method or combination of methods including biochemical, recombinant, and synthetic techniques. The polypeptide sequences may be prepared by at least one purification step.

The term “level” as used herein is intended to refer to the amount per weight or weight per weight of an analyte of interest, (e.g.) a troponin such as cTnI. It is also intended to encompass “concentration” expressed as amount per volume or weight per volume. The term “circulating level” is intended to refer to the amount per weight or weight per weight or concentration of, for example, cardiac troponins present in the circulating fluid such as plasma, serum or whole blood.

The term “marker C” as used herein is intended to mean IGFBP3.

The term “marker D” as used herein is intended to mean bTnI-IgG as defined, and is also referred to throughout as “bTnI”.

As used herein, the terms “manage”, “managing”, and “management” in the context of the administration of a therapy to a subject refer to the beneficial effects that a subject derives from a therapy (e.g., a prophylactic or therapeutic agent) or a combination of therapies, while not resulting in a cure of the disease or condition. In certain examples, a subject is administered one or more therapies (e.g., one or more prophylactic or therapeutic agents) to “manage” the disease or condition so as to prevent the progression or worsening of the disease or condition.

The term “MACE” as used herein means major acute cardiac event.

The terms “marker” or “biomarker” in the context of an analyte means any antigen, molecule or other chemical or biological entity that is specifically found in circulation or associated with a particular tissue (e.g. heart muscle) that it is desired to be identified in or on a particular tissue affected by a disease or disorder, for example unstable angina. In specific examples, the marker is a circulating cardiac troponin (e.g.) bTnI-IgG.

The terms “MI” and “AMI” as used herein mean (acute) myocardial infarction, a type of acute coronary syndrome.

The term “NCCP” as used herein means non-cardiac chest pain.

The term “NSTEMI” as used herein means non-ST elevation myocardial infarction, a type of myocardial infarction typically characterised by a depressed ST wave or T-wave inversion, no progression to Q wave and partial blockage of the coronary artery.

The term “NT-proBNP” as used herein means N-Terminal pro B-Type Natriuretic Peptide.

The terms “peptide” and “polypeptide” or “selectively binds” may be used interchangeably throughout this specification, and encompass amino acid chains of any length, including full length sequences in which amino acid residues are linked by covalent peptide bonds. Polypeptides useful in the present invention may be purified natural products, or may be produced partially or wholly using recombinant or synthetic techniques. The term “polypeptide” may refer to a polypeptide, an aggregate of a polypeptide such as a dimer or other multimer, a fusion polypeptide, a polypeptide fragment, a polypeptide variant, or derivative thereof. Polypeptides herein may have chain lengths of at least 40 amino acids, at least 50 amino acids, or at least 60, at least 70, at least 80, at least 90, at least 100, at least 110, at least 120, at least 130, at least 140, at least 150, at least 160, at least 170, at least 180, at least 190, at least 200, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250, at least 251, at least 252, at least 253, at least 254, at least 255, at least 256, at least 257, at least 258, at least 259, at least 260, at least 261, at least 262, at least 263, at least 264 amino acids. Reference to other polypeptides of the invention or other polypeptides described herein should be similarly understood.

The term “purified” as used herein does not require absolute purity. Purified refers in various embodiments, for example, to at least about 80%, 85%, 90%, 95%, 98%, or 99% homogeneity of a polypeptide, for example, in a sample. The term should be similarly understood in relation to other molecules and constructs described herein.

The term “Px” as used herein means prediction or prognostic.

Specifically, the term “reference interval” or “reference standard” as used herein is intended to refer to a figure within a statistical band of a representative concentration or alternatively a figure with an upper or lower concentration. The reference interval or reference standard will typically be obtained from subjects that do not have any pre-existing conditions that could result in changes to the level of circulating cardiac troponins.

The acronym “RiskDM” as used herein is intended to mean risk of diabetes or diabetes mellitus.

The term “ROC” means Receiver Operating Curve and a ROC plot depicts the overlap between two distributions by plotting the sensitivity versus 1-specificity for a complete range of decision thresholds.

The term “subject” or “patient” may be used interchangeably in this specification and it intended to refer to a human or non-human primate. In one example, the subject or patient is a human.

The terms “specifically binds” or “selectively binds” may be used interchangeably throughout this specification, and shall be taken to mean that the binding agent reacts or associates more frequently, more rapidly, with greater duration and/or with greater affinity to a particular substance than it does with alternative substances. For example, a binding agent that specifically binds to bTnI-IgG, as well as isoforms thereof, or an epitope or immunogenic fragment thereof with greater affinity, avidity, more readily, and/or with greater duration than it binds to unrelated protein and/or epitopes or immunogenic fragments thereof. It is also understood by reading this definition that, for example, a binding agent that specifically binds to a first target (e.g. bTnI-IgG) may or may not specifically bind to a second target. As such, “specific binding” does not necessarily require exclusive binding or non-detectable binding of another molecule. Generally, but not necessarily, reference to binding means specific binding.

In addition to computer/database methods known in the art, polypeptide antigenic variants may be identified by physical methods known in the art, for example, by screening expression libraries using antibodies raised against polypeptides of the invention (Sambrook et al., Molecular Cloning: A Laboratory Manual, 2nd Ed. Cold Spring Harbor Press, 1987) by recombinant DNA techniques also described by Sambrook et al. or by identifying polypeptides from natural sources with the aid of such antibodies.

The term “STEMI” as used herein means ST elevation myocardial infarction, a type of acute myocardial infarction typically characterised by elevated ST wave, progression to Q wave and full blockage of the coronary artery.

As used herein, the term “therapeutic agent” refers to any molecule, compound, and/or substance that is used for the purpose of treating and/or managing a disease or disorder, such as unstable angina. Examples of therapeutic agents include, but are not limited to, proteins, immunoglobulins (e.g., multi-specific Igs, single chain Igs, Ig fragments, polyclonal antibodies and their fragments, monoclonal antibodies and their fragments), peptides (e.g., peptide receptors, selectins), binding proteins, biologics, proliferation-based therapy agents, hormonal agents, radioimmunotherapies, targeted agents, epigenetic therapies, differentiation therapies, biological agents, and small molecule drugs.

As used herein, the terms “therapies” and “therapy” can refer to any method(s), composition(s), and/or agent(s) that can be used in the prevention, treatment and/or management of a disease or condition or one or more symptoms thereof.

The term “TID” as used herein mean transient ischaemia dilation, which may be confirmed, for example, using spectral imaging or ultrasound.

The term “TnT” means Troponin T, preferably derived from a cardiac source (i.e. and “cTnT”).

The term “bTnI-IgG” refers to a bound complex of cardiac troponin I (cTnI) and an immunoglobulin G (IgG) including, without limitation, IgG1, IgG2, IgG3 and IgG4.

As used herein, the terms “treat”, “treatment” and “treating” in the context of the administration of a therapy to a subject refer to the reduction, inhibition, elimination or amelioration of the progression and/or duration of (e.g.) an acute coronary syndrome, the reduction, inhibition, elimination or amelioration of the severity of (e.g.) acute coronary syndrome, and/or the amelioration of one or more symptoms thereof resulting from the administration of one or more therapies.

The term “UA” and “UAP” as used herein means unstable angina or unstable angina pectoris, a type of acute coronary syndrome.

The term “UDCP” as used herein means undifferentiated chest pain.

Term “variant” as used herein refers to polypeptide sequences different from the specifically identified sequences, wherein 1 to 6 or more or amino acid residues are deleted, substituted, or added. Substitutions, additions or deletions of one, two, three, four, five or six amino acids are contemplated. Variants may be naturally occurring allelic variants, or non-naturally occurring variants. Variants may be from the same or from other species and may encompass homologues, paralogues and orthologues. In certain embodiments, variants of the polypeptides useful in the invention have biological activities including signal peptide activity or antigenic-binding properties that are the same or similar to those of the parent polypeptides. The term “variant” with reference to polypeptides encompasses all forms of polypeptides as defined herein.

Variant polypeptide sequences exhibit at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to a sequence of the present invention. With regard to polypeptides, identity is found over a comparison window of at least 233 to 291 amino acid positions.

Polypeptide variants also encompass those which exhibit a similarity to one or more of the specifically identified sequences that is likely to preserve the functional equivalence of those sequences, including those which could not reasonably be expected to have occurred by random chance.

Polypeptide sequence identity and similarity can be determined in the following manner. The subject polypeptide sequence is compared to a candidate polypeptide sequence using BLASTP (from the BLAST suite of programs, version 2.2.18 [April 2008]]) in bl2seq, which is publicly available from NCBI (ftp://ftp.ncbi.nih.gov/blast/). The default parameters of bl2seq are utilized except that filtering of low complexity regions should be turned off.

The similarity of polypeptide sequences may be examined using the following UNIX command line parameters: bl2seq-i peptideseq1-j peptideseq2-F F-p blastp. The parameter-F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match. Variant polypeptide sequences commonly exhibit an E value of less than 1×10⁻⁵, less than 1×10⁻⁶, less than 1×10⁻⁹, less than 1×10⁻¹², less than 1×10⁻¹⁵, less than 1×10⁻¹⁸ or less than 1×10⁻²¹ when compared with any one of the specifically identified sequences. Polypeptide sequence identity may also be calculated over the entire length of the overlap between a candidate and subject polypeptide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235) as discussed above are also suitable global sequence alignment programs for calculating polypeptide sequence identity. Use of BLASTP is preferred for use in the determination of polypeptide variants according to the present invention.

### DETAILED DESCRIPTION

The troponin market is estimated to be worth in excess of US$1B.

Medical cardiac troponin T (cTnT) measurement is provided by one main supplier, Roche Diagnostics. Cardiac troponin I (cTnI) measurements are provided by more than twenty suppliers. Accordingly, cTnT measurements benefit from standardization from one supplier whereas cTnI measurement does not.

Despite these observations, auto-antibodies produced by the body can interfere with the binding sites that both cTnT and cTnI tests utilise, especially in patients who present with symptoms of myocardial infarction and a history of cardiovascular disease. This can lead to false low values, potentially missing a heart attack diagnosis. Conversely, false high values can be found as auto-antibody bound troponin has reduced clearance from the body. The prevalence of this is ~10% in healthy individuals and up to 30% of those with cardiovascular disease, but prior identification of individuals is difficult meaning the entire population would need to be tested in an acute setting.

An issue not contemplated by existing commercial troponin assays is the interference caused by troponin-troponin interactions (i.e.) cTnI directly binding to cTnT and influencing measured levels. In other words, the combination of cTnI-cTnT interactions and auto-antibody levels to influence diagnostic troponin assay performance has not been addressed.

The Applicant has therefore conceived, designed and developed a clinically meaningful test (two-hour turn-around) that measures auto-antibody bound cTnI, free from cTnT interactions.

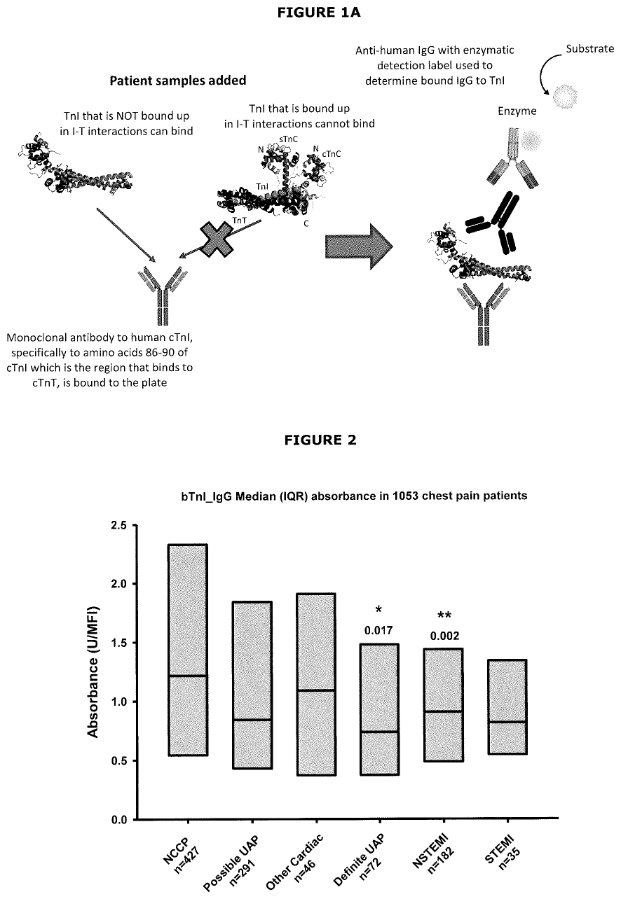

Specifically, and with reference to the assay concept illustrated in FIG. 1, the Applicant was able to measure free/non-troponin bound cTnI-Ab complex (i.e. not bound to cTnT) in a sandwich ELISA which utilises an anti-cTnI antibody as a capture antibody and an anti-IgG antibody as a detection antibody. This simplistic but effective approach enables the measurement of a bound complex comprising cTnI and IgG, referred to herein as “bTnI-IgG”. Importantly, bTnI-IgG and may be used to as an adjunct biomarker to reflect the amount of cTnI bound to cTnT (i.e.) reduced levels of bTnI-IgG measured relative to a reference standard from a control population would reflect increased amounts of cTnI that is bound to cTnT in a TnI-TnT complex. In other words, lower test values in the bTnI-IgG assay reflect a greater proportion of cTnI that is bound up in TnI-TnT complexes, which is more likely to be present during an acute cardiac event.

Accordingly, in an aspect of the present invention there is provided a method for determining the level of a bound protein complex comprising cardiac Troponin I (cTnI) and an immunoglobulin G (IgG) (bTnI-IgG) in a biological sample obtained from a patient, the method comprising:
(i) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
(ii) washing the reaction mix from (i) to remove non-selectively bound analytes;
(iii) contacting the reaction mix from (ii) with an anti-IgG detection antibody, for a time and under conditions sufficient for the anti-IgG detection antibody to selectively bind to the bTnI-IgG complex; and
(iv) determining the level of bTnI-IgG in the biological sample.

In an example according to this and other aspects of the present invention, the anti-cTnI capture antibody selectively binds to an equivalent region comprising amino acids 86-90 of cTnI as defined by SEQ ID NO: 1.

In another example according to this and other aspects of the present invention, the anti-cTnI capture antibody is a monoclonal anti-cTnI antibody.

In a further example according to this and other aspects of the present invention, the anti-IgG detection antibody is a human anti-IgG antibody.

In another example according to this and other aspects of the present invention, the anti-cTnI monoclonal antibody is produced by Hytest, Abcam, NovusBio, ThermoFisher, Biotechne.

In yet a further example according to this and other aspects of the present invention, the anti-cTnI monoclonal antibody is immobilized on a solid substrate. Specific examples of solid substrates are provided in more detail below.

In yet another example according to this and other aspects of the present invention, the anti-IgG antibody, including anti-human IgG antibody, comprises a detectable label. In a related example, the detectable label is an enzymatic detection label.

In yet another example according to this and other aspects of the present invention, the biological sample is selected from plasma, serum, whole blood, arterial blood, venous blood, saliva, bone marrow tissue, heart tissue, vascular tissue and interstitial fluid sample.

In a related example according to this and other aspects of the present invention, the biological sample is a circulating sample selected from plasma, serum, whole blood, arterial blood and venous blood.

In a further related example according to this and other aspects of the present invention, the biological sample is plasma.

In yet a further example according to this and other aspects of the present invention, step (a) is performed at room temperature for about one hour.

A person skilled in the art would recognise the term “about 1 hour” to mean a period of time which is about an hour and includes, without limitation, about 55, 56, 57, 58, 59, 60, 61, 62, 63, 64 and 65 minutes, although by way of illustration (e.g.) 50 and 70 minutes would still meet this definition.

In yet a further example according to this and other aspects of the present invention, step (b) comprises a first wash and a second wash, wherein the first and second washes are spaced apart by about 30 minutes.

A person skilled in the art would recognise the term “about 30 minutes” to mean a period of time which is about half an hour and includes, without limitation, about 25, 26, 27, 28, 29, 30, 31, 32, 33, 34 and 35 minutes, although by way of illustration (e.g.) 20 and 40 minutes would still meet this definition.

In another example according to this and other aspects of the present invention, the level of bTnI-IgG in the biological sample is between about 0.01 and 100 ug/mL, and includes without limitation about 0.01, 0.05, 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, 90, 95 or about 100 ug/mL.

In another example according to this and other aspects of the present invention, the method further comprises comparing the measured level of bTnI-IgG level against a reference standard from a control population.

In a related example, the reference standard is from a control population of sex and age-matched subjects who have not been identified as having an acute coronary syndrome or a history of an acute coronary syndrome.

The hypothesis that lower levels of bTnI-IgG (i.e. relevant to a reference standard from a control population) are more likely to reflect an acute cardiac event was subsequently validated using clinical samples derived from the ‘SPACE’ patient cohort (Christchurch School of Medicine, New Zealand; refer to Example 2 for further details). The data presented in FIG. 2 shows median interquartile ranges of bTnI-IgG were lower in patients with an adjudicated diagnosis of an acute coronary syndrome (e.g.) STEMI, NSTEMI and UAP, as compared to patients with an adjudicated diagnosis of non-acute (e.g. “other cardiac issues”) or non-cardiac issues (“NCCP”).

Importantly, the data presented in Table 1 below also reflects a negative relationship between bTnI-IgG and NT-proBNP according to Spearman's rank correlation coefficient analysis.

TABLE 1
Spearman's Rho Analysis

| Biomarker | Spearman's rho | P Value |
|---|---|---|
| bTnI-IgG | 1 | — |
| hsTnI | −0.189 | <0.001 |
| hsTnT | 0.058 | 0.068 |
| NT-proBNP | −0.432 | <0.001 |

Accordingly, in a further aspect of the present invention there is provided a method for diagnosing an acute coronary syndrome in a patient, the method comprising:
(i) determining the level of a bound protein complex comprising cardiac Troponin I (cTnI) and an immunoglobulin G (IgG) (bTnI-IgG) in a biological sample obtained from the patient which comprises:
   (a) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
   (b) washing the reaction mix from (a) to remove non-selectively bound analytes;
   (c) contacting the reaction mix from (b) with an anti-IgG detection antibody, for a time and under conditions sufficient for the anti-IgG detection antibody to selectively bind to the bTnI-IgG complex; and
   (d) determining the level of bTnI-IgG in the biological sample;
(ii) comparing the level of bTnI-IgG from (i) against a reference standard from a control population
wherein, a lower level of bTnI-IgG in the biological sample measured relative to the reference standard from a control population is indicative that the patient has an acute coronary syndrome.

In an example according to this and other aspects of the present invention, the acute coronary syndrome is selected from unstable angina or unstable angina pectoris; cardiac ischemia and myocardial ischemia; Type 1 and Type 2 (acute) myocardial infarction including ST-elevation myocardial infarction and non-ST myocardial infarction; acute cardiac injury; acute cardiac damage resulting from acute drug toxicity, acute cardiomyopathies and cardiac transplant rejection.

In a related example, the acute coronary syndrome is acute myocardial infarction or unstable angina pectoris.

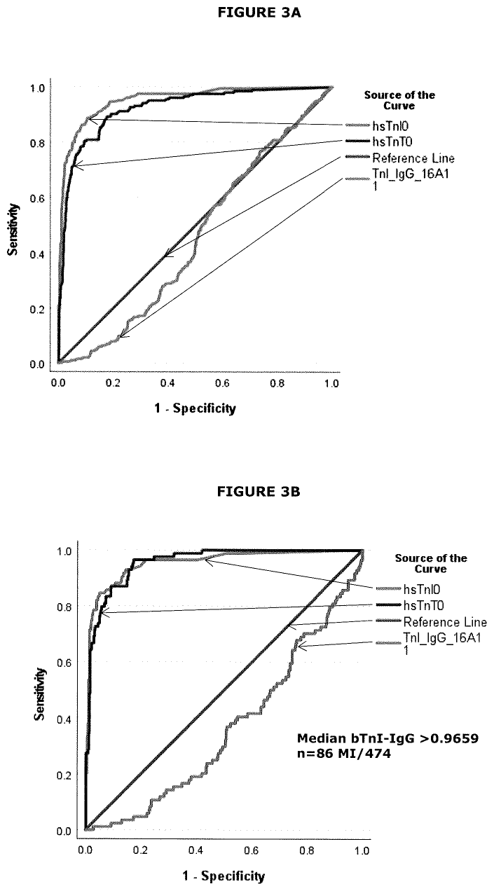

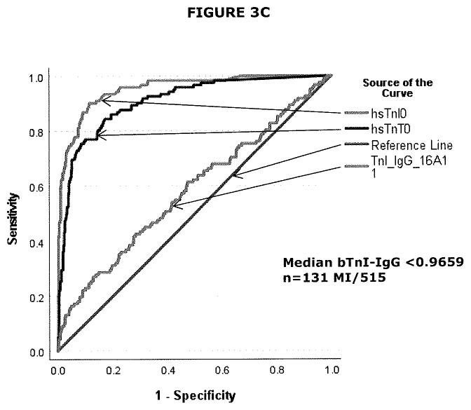

The clinical significance of the bTnI-IgG assay developed by the Applicant is further realised by the data presented in Example 4 and FIGS. 3-5 for the diagnosis of acute myocardial infarction. Specifically, using Receiver Operating Curve analysis, the performance of hsTnT was enhanced by its combination with bTnI-IgG where the area under the curve increased from 0.76 (hsTnT) to 0.81 (hsTnT+bTnI-IgG) according to the analyses performed. Given the log scale associated with ROCs, a person skilled in the art would recognise that this increase represents a statistically significant improvement for the diagnosis of an acute myocardial infarction. Indeed, according to the data presented in Table 4 of Example 4, the specificity of the hsTnT assay is significantly improved by the presence of bTnI-IgG where the specificity increased from 63% to 76% when the sensitivity was held at 79%.

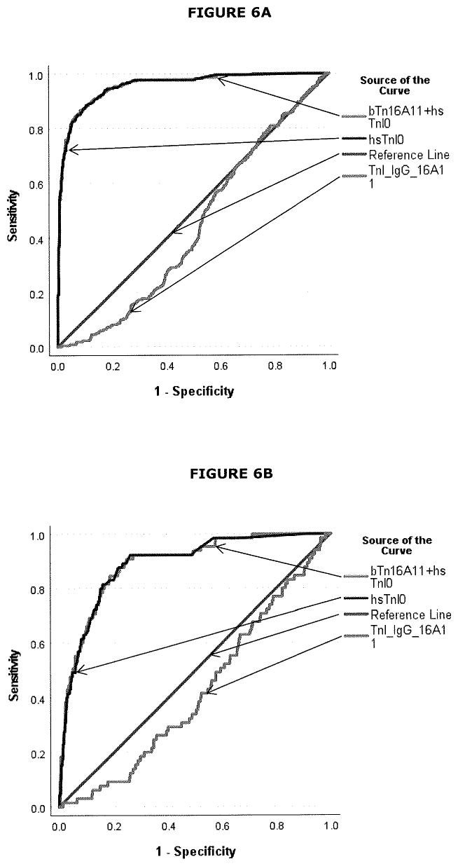

These data are further supported by the results presented in Example 5 and FIGS. 6A and 6B, where the Applicant showed that bTnI-IgG does not enhance the performance of hsTnI for the diagnosis of myocardial infarction. This result is to be expected given that the assay according to the present invention is intended to measure free/unbound TnI as a proxy to the amount of TnT bound to TnI-TnT complexes.

Accordingly, in another aspect of the present invention there is provided a method for enhancing the diagnostic performance of a cardiac troponin T (cTnT) assay for the diagnosis of an acute myocardial infarction in a patient, the method comprising:
(i) determining the level of a bound protein complex comprising cardiac Troponin I (cTnI) and an immunoglobulin G (IgG) (bTnI-IgG) in a biological sample obtained from the patient which comprises:
   (a) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
   (b) washing the reaction mix from (a) to remove non-selectively bound analytes;
   (c) contacting the reaction mix from (b) with an anti-IgG detection antibody, for a time and under conditions sufficient for the anti-IgG detection antibody to selectively bind to the bTnI-IgG complex; and
   (d) determining the level of bTnI-IgG in the biological sample;
(ii) comparing the level of bTnI-IgG from (i) against a reference standard from a control population
wherein, when the level of bTnI-IgG in the biological sample is lower compared to the level of bTnI-IgG of a reference standard from a control population it is combined with cTnT to improve the diagnostic accuracy of cTnT which is achieved in the absence of bTnI-IgG.

In an example according to this and other aspects of the present invention, the cTnT is highly sensitive cardiac troponin T (hsTnT).

In another example according to this and other aspects of the present invention, the concentration of cTnT in the patient sample is less than or equal to 15 ng/L, for example 14, 13, 12, 11, 10, 9, 8, 7, 6, 5, 4, 3, 2 or 1 ng/L.

In another example according to this and other aspects of the present invention, the level of bTnI-IgG in the patient sample is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or at least 10 times lower than the level of bTnI-IgG in the reference standard from a control population.

In another example according to this aspect of the present invention, the level of bTnI-IgG in the patient sample is at least 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 times lower than the level of bTnI-IgG in the reference standard from a control population.

In another example according to this and other aspects of the present invention, the reference standard is from a control population of sex and age-matched subjects who have not been identified as having acute myocardial infarction or a history of acute myocardial infarction.

In other examples according to any of the methods described herein, the concentration or level of bTnI-IgG is obtained from a biological sample taken from a patient at index presentation. This includes, without limitation, at a time when a patient (e.g.) presents to an emergency department, a clinic, a hospital, a surgery, a doctor's practice, a doctor or any other relevant medical forum, and information about the cardiac status of the patient is measured, including the patient's bTnI-IgG levels. Alternatively index presentation means a time when a patient identifies or presents with a complaint of chest pain during the hospital admission period, during a follow-up consultation or during participation in a clinical trial. The term “0” in the tables and figures which follow (e.g. “hsTnT0”) is intended to mean the levels of highly sensitive troponin T an index presentation, for example.

In another example according to these and other aspects of the present invention, multiple samples are taken from a patient while in the acute clinical setting. By way of illustration only, a first sample and second sample may be obtained from a patient and the time point between obtaining the first sample and second samples may be between about half an hour to about 10 hours. This includes, without limitation, about 0.5 h, about 1 h, about 1.5 h, about 2 h, about 2.5 h, about 3 h, about 3.5 h, about 4 h, about 4.5 h, about 5 h, about 5.5 h, about 6 h, about 6.5 h, about 7 h, about 7.5 h, about 8 h, about 8.5 h, about 9 h, about 9.5 h or about 10 h.

In further examples according to the methods described herein, bTnI-IgG levels may be measured together with one or more other cardiac risk factors including, without limitation, a history of cardiovascular disease (e.g. HxMI or HxUAP), an increased heart rate relevant to a reference standard, an abnormal electrocardiogram, a decreased level of high-density lipoprotein relevant to a reference standard, a diagnosis of ischaemia, optionally by imaging, or dyslipidemia or a history of dyslipidemia, in isolation or in any combination.

A person skilled in the art would appreciate the terms “measured together with” or “measured together with one or more risk factors” in the context of (e.g.) the clinical risk factors identified above is intended to mean that (i) the patient has presented with one or more of those risk factors (ii) or it is determined that one or more of those risk factors exist when interrogating bTnI-IgG levels.

Advantageously, the diagnosis of an acute coronary syndrome made in accordance with methods described herein may be useful to inform a therapeutic regime to control, reverse, mitigate or treat ACS in the patient.

As such, in a further aspect of the present invention there is provided a method for treating an acute coronary syndrome in a patient, the method comprising:
(i) determining the level of a bound protein complex comprising cardiac Troponin I (cTnI) and an immunoglobulin G (IgG) (bTnI-IgG) in a biological sample obtained from the patient which comprises:
   (a) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
   (b) washing the reaction mix from (a) to remove non-selectively bound analytes;
   (c) contacting the reaction mix from (b) with an anti-IgG detection antibody, for a time and under conditions sufficient for the anti-IgG detection antibody to selectively bind to the bTnI-IgG complex; and
   (d) determining the level of bTnI-IgG in the biological sample;
(ii) comparing the level of bTnI-IgG from (i) against a reference standard from a control population wherein a lower level of bTnI-IgG in the biological sample compared to the reference standard from the control population is indicative that the patient has an acute coronary syndrome; and
(iii) where the level of bTnI-IgG in the biological sample is lower than the reference standard from the control population, triaging and/or treating the human for the acute coronary syndrome.

The therapeutic regimes administered in accordance with the methods of the present invention include, by way of illustration and example only, those outlined by the Mayo Clinic in its medication guidelines:
https://www.mayoclinic.org/diseases-conditions/myocardial-ischemia/diagnosis-treatment/drc-20375422.

By way of illustration only, the therapeutic regimes administered in accordance with the present invention include, without limitation, administration of one or more drugs selected from the group consisting of aspirin, nitrates, beta-blockers, calcium channel blockers, cholesterol lowering medications, angiotensin-converting enzyme inhibitors, ranolazine, and any combination thereof.

Finally, the Applicant has also identified a possible utility for bTnI-IgG to predict future (e.g.) ACS event, positive stress test, stroke or mortality. Further information is provided in Examples 6-14, read in conjunction with Figures and taken in the context of the Summary of the Invention provided previously.

In particular, the present invention further provides a method for predicting a new acute coronary syndrome in a patient, the method comprising:
(i) determining the level of a bound protein complex comprising cardiac Troponin I (cTnI) and an immunoglobulin G (IgG) (bTnI-IgG) in a biological sample obtained from the patient which comprises:
   (a) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
   (b) washing the reaction mix from (a) to remove non-selectively bound analytes;
   (c) contacting the reaction mix from (b) with an anti-IgG detection antibody, for a time and under conditions sufficient for the anti-IgG detection antibody to selectively bind to the bTnI-IgG complex; and
   (d) determining the level of bTnI-IgG in the biological sample;
(ii) comparing the level of bTnI-IgG from (i) against a reference standard from a control population
wherein, a lower level of bTnI-IgG in the biological sample measured relative to the reference standard from a control population is indicative that the patient has an acute coronary syndrome.

In an example according to this and other aspects of present invention in the context of predicting an acute coronary syndrome, the patient did not have a diagnosis of myocardial infarction at index presentation.

In another example according to this and other aspects of present invention in the context of predicting an acute coronary syndrome, the patient did have a diagnosis of myocardial infarction at index presentation.

In a further example according to this and other aspects of present invention in the context of predicting an acute coronary syndrome, bTnI-IgG was used to predict new ACS within about 1 year or about 365 days.

In yet a further example according to this and other aspects of present invention in the context of predicting an acute coronary syndrome, bTnI-IgG was determined alongside other clinical factors including but not limited to a significant history of cardiovascular disease (including (e.g.) an index diagnosis of myocardial infarction, an index diagnosis of Type 2 myocardial infarction, an index diagnosis of unstable angina pectorus), radiology findings of artery occlusion, diastolic blood pressure, IGFBP3, risk of diabetes mellitus, abnormal cholesterol, abnormal hemaglobin level and includes any combination thereof. These clinical variable may collectively be included in various regression modelling to establish the strength of correlation as will be known to a person skilled in the art.

### Antibodies and Antigen Binding Fragments

The present invention contemplates various antibodies and antigen-binding fragments thereof which selectively bind to cardiac troponins or antigenic variants of cardiac troponins including isoforms thereof. The present invention also contemplates various antibodies and antigen-binding fragments thereof which selectively bind to an immunoglobulin (IgG) bound to cardiac troponin I or cardiac troponin T.

Accordingly, in yet another aspect of the present invention there is provided an antibody or antigen-binding fragment which selectively binds to cardiac troponin I or cardiac troponin T. In yet another aspect the present invention alternatively provides an antibody or antigen-binding fragment which selectively binds to an immunoglobulin (IgG) bound to cardiac troponin I or cardiac troponin T.

In yet a further aspect of the present invention there is provided a monoclonal antibody, a polyclonal antibody, a chimeric antibody or a humanized antibody which selectively binds to cardiac troponin I or cardiac troponin T, or an antigen-binding fragment of a monoclonal, polyclonal, chimeric or humanized antibody which selectively binds to cardiac troponin I or cardiac troponin T. In yet a further aspect the present invention alternatively provides a monoclonal antibody, a polyclonal antibody, a chimeric antibody or a humanized antibody which selectively binds to an immunoglobulin (IgG) bound to cardiac troponin I or cardiac troponin T.

In yet another aspect of the present invention there is provided a monoclonal antibody or antigen-binding fragment thereof which selectively binds to cardiac troponin I or cardiac troponin T. In yet another aspect the present invention alternatively provides a monoclonal antibody which selectively binds to an immunoglobulin (IgG) bound to cardiac troponin I or cardiac troponin T.

In yet a further aspect of the present invention there is provided an antibody or antigen-binding fragment which selectively binds to cardiac troponin I or cardiac troponin T, which antibody or antigen-binding fragment comprises a detectable label. In yet a further aspect of the present invention alternatively provides an antibody or antigen-binding fragment which selectively binds to an immunoglobulin (IgG) bound to cardiac troponin I or cardiac troponin T.

In yet another aspect of the present invention there is provided an antibody or antigen-binding fragment which selectively binds to cardiac troponin I or cardiac troponin T, which antibody or antigen-binding fragment is immobilized on a solid substrate. In yet another aspect the present invention alternatively provides an antibody or antigen-binding fragment which selectively binds to an immunoglobulin (IgG) bound to cardiac troponin I or cardiac troponin T, which antibody or antigen-binding fragment is immobilized on a solid substrate.

In yet a further aspect of the present invention there is provided a binding agent comprising a peptide framework comprising one or more complementarity determining regions derived from an antibody which selectively binds to cardiac troponin I or cardiac troponin T. In yet a further aspect the present invention alternatively provides a binding agent comprising a peptide framework comprising one or more complementarity determining regions derived from an antibody which selectively binds to an immunoglobulin (IgG) bound to cardiac troponin I or cardiac troponin T.

In yet a further aspect of the present invention there is provided a binding agent comprising a peptide framework comprising at least three complementarity determining regions derived from an antibody which selectively binds to cardiac troponin I or cardiac troponin T. In yet a further aspect the present invention alternatively provides a binding agent comprising a peptide framework comprising at least three complementarity determining regions derived from an antibody which selectively binds to an immunoglobulin (IgG) bound to cardiac troponin I or cardiac troponin T.

As noted above, antibody or antibodies as used herein refers to a peptide or polypeptide derived from, modelled after or substantially encoded by an immunoglobulin gene or immunoglobulin genes, or fragments thereof, capable of specifically binding an antigen or epitope. As foreshadowed in the definition section of this specification, the term antibody includes antigen binding fragments such as, for example, fragments, subsequences, complementarity determining regions (CDRs) that retain capacity to bind to an antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; and (vi) an isolated complementarity determining region (CDR). Single chain antibodies are also included by reference in the term “antibody.”

Also included is antiserum obtained by immunizing an animal such as a mouse, rat or rabbit with an antigen, such as for example, cardiac troponin I or cardiac troponin T. In brief, methods of preparing polyclonal antibodies are known to a person skilled in the art. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include cardiac troponin I or cardiac troponin T, antigenic variants thereof or a fusion protein thereof. It may be useful to conjugate the immunizing agent to a protein known to be immunogenic in the mammal being immunized. Examples of such immunogenic proteins include but are not limited to keyhole limpet hemocyanin, bovine serum albumin, bovine thyroglobulin, and soybean trypsin inhibitor. Examples of adjuvants that may be employed include Freund's complete adjuvant and MPL TDM adjuvant (monophosphoryl Lipid A, synthetic trehalose dicorynomycolate). The immunization protocol may be selected by one skilled in the art without undue experimentation.

Monoclonal antibodies may be prepared using hybridoma methods well known in the art. The hybridoma cells may be cultured in a suitable culture medium, alternatively, the hybridoma cells may be grown in vivo as ascites in a mammal. Preferred immortalized cell lines are murine myeloma lines, which can be obtained, for example, from the American Type Culture Collection, Virginia, USA. Immunoassays may be used to screen for immortalized cell lines that secrete the antibody of interest. Sequences of cardiac troponin I or cardiac troponin T or antigenic variants thereof may be used in screening.

Well known means for establishing binding specificity of monoclonal antibodies produced by the hybridoma cells include immunoprecipitation, radiolinked immunoassay (RIA), enzyme-linked immunoabsorbent assay (ELISA) and Western blot. For example, as noted above, the binding affinity of the monoclonal antibody can, for example, be determined by the Scatchard analysis. Samples from immunised animals may similarly be screened for the presence of polyclonal antibodies.

Monoclonal antibodies can also be obtained from recombinant host cells. DNA encoding the antibody can be obtained from a hybridoma cell line. The DNA is then placed into an expression vector, transfected into host cells (e.g., COS cells, CHO cells, E. coli cells) and the antibody produced in the host cells. The antibody may then be isolated and/or purified using standard techniques.

The monoclonal antibodies or fragments may also be produced by recombinant DNA means. DNA modifications such as substituting the coding sequence for human heavy and light chain constant domains in place of the homologous murine sequences are also possible. The antibodies may be monovalent antibodies. Methods for preparing monovalent antibodies are well known in the art. Production of chimeric, bivalent antibodies and multivalent antibodies are also contemplated herein.

Other known art techniques for monoclonal antibody production such as from phage libraries, may also be used.

The monoclonal antibodies secreted by the cells may be isolated or purified from the culture medium or ascites fluid by conventional immunoglobulin purification procedures such as, for example, reverse phase HPLC, protein A-Sepharose, hydroxyapatite chromatography, gel electrophoresis, dialysis, or affinity chromatography.

Bispecific antibodies may also be useful. These antibodies are monoclonal, preferably human or humanized, antibodies that have binding specificities for at least two different antigens. Antibodies with greater than two specificities for example trispecific antibodies are also contemplated herein.

Antibodies used in the immunoassays described herein selectively bind to IGFBP-3. The term “selectively binds” is not intended to indicate that an antibody binds exclusively to its intended target since, as noted above, an antibody binds to any polypeptide displaying the epitope(s) to which the antibody binds. Rather, an antibody “selectively binds” if its affinity for its intended target is about 5-fold greater when compared to its affinity for a non-target molecule which does not display the appropriate epitope(s). In certain examples, the affinity of the antibody will be at least about 5 fold, preferably 10 fold, more preferably 25-fold, even more preferably 50-fold, and most preferably 100-fold or more, greater for a target molecule than its affinity for a non-target molecule. In other examples, antibodies bind with affinities of at least about 10⁻⁶M, or 10⁻⁷M, or at least about 10⁻⁸M, or 10⁻⁹M, or 10⁻¹⁰ M, or 10⁻¹¹M or 10⁻¹²M.

Affinity is calculated as K_{d}=k_{off}/kₒₙ (k_{off} is the dissociation rate constant, Kₒₙ is the association rate constant and K_{d} is the equilibrium constant). Affinity can be determined at equilibrium by measuring the fraction bound (r) of labelled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art.

Numerous publications discuss the use of phage display technology to produce and screen libraries of polypeptides for binding to a selected analyte. A basic concept of phage display methods is the establishment of a physical association between DNA encoding a polypeptide to be screened and the polypeptide. This physical association is provided by the phage particle, which displays a polypeptide as part of a capsid enclosing the phage genome that encodes the polypeptide. The establishment of a physical association between polypeptides and their genetic material allows simultaneous mass screening of very large numbers of phage bearing different polypeptides. Phage displaying a polypeptide with affinity to a target binds to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labelled secondary antibody (for example, an anti-mouse antibody conjugated to alkaline phosphatase if the raised antibodies are mouse antibodies) is added to the wells and incubated for about 30 min and then washed. Substrate is added to the wells and a colour reaction will appear where antibody to the immobilized polypeptide(s) is present.

The antibodies so identified may then be further analysed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interact with one another sterically, etc., assay performance of an antibody may be a more important measure than absolute affinity and specificity of an antibody.

### Aptamers

The present invention also contemplates aptamers that selectively bind to cardiac troponin I or cardiac troponin T or antigenic variants of cardiac troponin I or cardiac troponin T including isoforms thereof.

In yet another aspect of the present invention there is provided an aptamer or aptamer ligand binding domain which selectively binds to cardiac troponin I or cardiac troponin T, or to an immunoglobulin associated with cardiac troponin I or cardiac troponin T. In yet another aspect the present invention alternatively provides an aptamer or aptamer ligand binding domain which binds to an immunoglobulin (IgG) bound to a cardiac troponin I or cardiac troponin T.

Nucleic acid aptamers are nucleic acid molecules that have been engineered through repeated rounds of in vitro selection, SELEX (systematic evolution of ligands by exponential enrichment) to bind to various molecular targets such as small molecules, proteins, nucleic acids, and even cells, tissues and organisms. Aptamers offer molecular binding and recognition equivalent to antibodies. In addition to their discriminate recognition, aptamers offer advantages over antibodies as they can be engineered completely in vitro, are readily produced by chemical synthesis, possess desirable storage properties, and elicit little or no immunogenicity in therapeutic applications.

According to an example of the present invention, the aptamer is a monomer (one unit). According to another example of the invention, the aptamer is a multimeric aptamer. The multimeric aptamer may comprise a plurality of aptamer units (mers). Each of the plurality of units of the aptamer may be identical. In such a case the multimeric aptamer is a homomultimer having a single specificity but enhanced avidity (multivalent aptamer).

Alternatively, the multimeric aptamer may comprise two or more aptameric monomers, wherein at least two mers of the multimeric aptamer are non-identical in structure, nucleic acid sequence or both. Such a multimeric aptamer is referred to herein as a heteromultimer.

The heteromultimer may be directed to a single binding site i.e., monospecific (such as to avoid steric hindrance). The heteromultimer may be directed to a plurality of binding sites i.e., multispecific. The heteromultimer may be directed to a plurality of binding sites on different analytes. Further description of a multimeric aptamer is provided below.

A plurality of multimeric aptamers may be conjugated to form a conjugate of multimeric aptamers. The multimeric aptamer may comprise, two (dimer), three (trimer), four (tetramer), five (pentamer), six (hexamer), and even more units.

Aptamers of the invention can be synthesized and screened by any suitable methods in the art.

For example, aptamers can be screened and identified from a random aptamer library by SELEX (systematic evolution of ligands by exponential enrichment). Aptamers that bind to an antigen of interest can be suitably screened and selected by a modified selection method herein referred to as cell-SELEX or cellular-SELEX.

A random aptamer library can be created that contains monomeric, dimeric, trimeric, tetrameric or other higher multimeric aptamers. A random aptamer library (either ssDNA or RNA) can be modified by including oligonucleotide linkers to link individual aptamer monomers to form multimeric aptamer fusion molecules. In other examples, a random oligonucleotide library is synthesized with randomized 45 nt sequences flanked by defined 20 nt sequences both upstream and downstream of the random sequence, i.e., known as 5′-arm and 3′-arm, which are used for the amplification of selected aptamers. A linking oligonucleotide (i.e., linker) is designed to contain sequences complementary to both 5′-arm and 3′-arm regions of random aptamers to form dimeric aptamers. For trimeric or tetrameric aptamers, a small trimeric or tetrameric (i.e., a Holiday junction-like) DNA nanostructure is engineered to include sequences complementary to the 3′-arm region of the random aptamers, therefore creating multimeric aptamer fusion through hybridization. In addition, 3-5 or 5-10 dT rich nucleotides can be engineered into the linker polynucleotides as a single stranded region between the aptamer-binding motifs, which offers flexibility and freedom of multiple aptamers to coordinate and synergize multivalent interactions with cellular ligands or receptors. Alternatively, multimeric aptamers can also be formed by mixing biotinylated aptamers with streptavidin.

A modified cellular SELEX procedure can be employed to select target-binding aptamers. Multimeric aptamers may be multivalent but be of single binding specificity (i.e., homomultimeric aptamers). Alternatively, the multimeric aptamer may be multivalent and multi-specific (i.e., heteromultimeric aptamers).

Thus, each monomer of the homomultimeric aptamer binds the target protein (e.g., IGFBP-3 as well as antigenic variants thereof) in an identical manner. Thus according to an example of the invention, all monomeric components of the homomultimeric aptamer are identical.

Conversely, a heteromultimeric aptamer comprises a plurality of monomeric aptamers at least two of which bind different sites on a single target protein or bind at least two different target proteins.

Selection of RNA-aptamers is well-established using protocols described in the scientific literature.

In certain examples, a suitable nucleotide length for an aptamer ranges from about 15 to about 100 nucleotide (nt), and in various other examples, 12-30, 14-30, 15-30 nt, 30-100 nt, 30-60 nt, 25-70 nt, 25-60 nt, 40-60 nt, or 40-70 nt in length.

Multimerization can be done at the library level as follows.

In certain examples, a linker polynucleotide has a length between about 5 nucleotides (nt) and about 100 nt; in various examples, 10-30 nt, 20-30 nt, 25-35 nt, 30-50 nt, 40-50 nt, 50-60 nt, 55-65 nt, 50-80 nt, or 80-100 nt. It is within the ability of one of skill in the art to adjust the length of the linker polynucleotide to accommodate each monomeric aptamer in the multimeric structure.

In certain examples, the multimeric aptamers can be identified and screened from a random multimeric aptamer library as described herein. In other examples, the monomeric aptamers are linked to each other by one or a plurality of linker polynucleotides to form multimeric aptamers. Monomeric aptamers can be linked to form multimeric aptamers by any suitable means and in any configurations.

It will be appreciated that the monomeric structures of the invention can be further multimerized by post SELEX procedures.

Multimers can be linearly linked by continuous linear synthesis of DNA without spacers or with nucleic acid spacers. Aptamer synthesis usually relies on standard solid phase phosphoramitide chemistry.

Thus, dimers, trimers and tetramers or higher oligomeric structures (e.g., pentamers, hexamers, heptamers, octamers etc.) can be linked by a polymeric spacer.

In certain examples, the aptamers are further modified to protect the aptamers from nuclease and other enzymatic activities. The aptamer sequence can be modified by any suitable methods known in the art. For example, phosphorothioate can be incorporated into the backbone, and 5′-modified pyrimidine can be included in 5′ end of ssDNA for DNA aptamer. For RNA aptamers, modified nucleotides such as substitutions of the 2′—OH groups of the ribose backbone, e.g., with 2′-deoxy-NTP or -fluoro-NTP, can be incorporated into the RNA molecule using T7 RNA polymerase mutants. The resistance of these modified aptamers to nuclease can be tested by incubating them with either purified nucleases or nuclease from mouse serum, and the integrity of aptamers can be analyzed by gel electrophoresis.

The monomeric or multimeric aptamer of the invention can be further attached or conjugated to a detectable or therapeutic moiety (i.e., a pharmaceutical moiety).

Thus, as noted above, a diagnostic or therapeutic moiety can be attached to an aptamer embodied herein to provide additional biological activity, such as for diagnosing, preventing, or treating a condition or disease. In one example a diagnostic moiety such as a detectable moiety e.g., label (e.g., His tag, flag tag), fluorescent, radioactive, biotin/avidin etc., can be bound to the aptamer, and imaging, immunohistochemistry, or other invasive or non-invasive methods used to identify the location(s) and extend of binding of the conjugate to locations within the body. For therapeutic uses, a cytotoxic agent such as a chemotherapeutic agent, radioactive moiety, toxin, antibody, nucleic acid silencing agents e.g., small interferring RNA (siRNA) or other molecule with therapeutic activity when delivered to cells expressing a molecule to which the aptamer is targeted, may be used to enhance the therapeutic activity of the aptamer or provide a biological activity where the aptamer is providing the targeting activity. Moreover, other conjugates to the aptamers described herein are contemplated, such as but not limited to scaffolds, sugars, proteins, antibodies, polymers, and nanoparticles, each of which have art-recognized therapeutic or diagnostic utilities and can be targeted to particular sites in vivo using an aptamer embodied herein.

### Detection of Binding Agents Including Peptide Binding Assays

The present invention includes use of a detection system involving the binding bTnI-IgG to capture/detection antibodies so as to measure the amount of bTnI-IgG present in a biological sample under interrogation. A similar solution is to detect the amount of unbound binding agent in a sample to get an indication of unbound or bound bTnI-IgG. It is intended that such alternative methods fall within the scope of the present invention as functional alternatives to directly detecting the amount of bound binding agent. Persons skilled in the art will appreciate that the concentration of bTnI-IgG in a sample can be readily calculated from the amount of bTnI-IgG in a sample when the sample volume is known.

In the assays, methods and kits according to the present invention, the measuring steps comprise detecting binding between bTnI-IgG and a binding agent that binds, selectively or specifically, to bTnI-IgG, and has low cross-reactivity with other markers of biological events.

In certain examples, the binding agent is an antibody or an antigen-binding fragment thereof. The antibody may be a monoclonal, polyclonal, chimeric or humanized antibody or antigen-binding fragment thereof. As such, in one example the assay, as well as methods involving assays, of the present invention is an immunoassay.

The antibodies of the present invention are particularly useful in immunoassays for determining the presence and/or amount of bTnI-IgG in a sample. Due to variable binding affinities of different antibodies, the person skilled in the art will appreciate that a standard binding curve of measured values versus amount of bTnI-IgG in a sample should be established for a particular antibody to enable the amount of bTnI-IgG in a sample to be determined. Such a curve is used to determine the true amount of bTnI-IgG in a sample.

Sample materials include biological fluids but are not limited thereto. In terms of the present invention, biological fluids are typically selected from whole blood, plasma or serum.

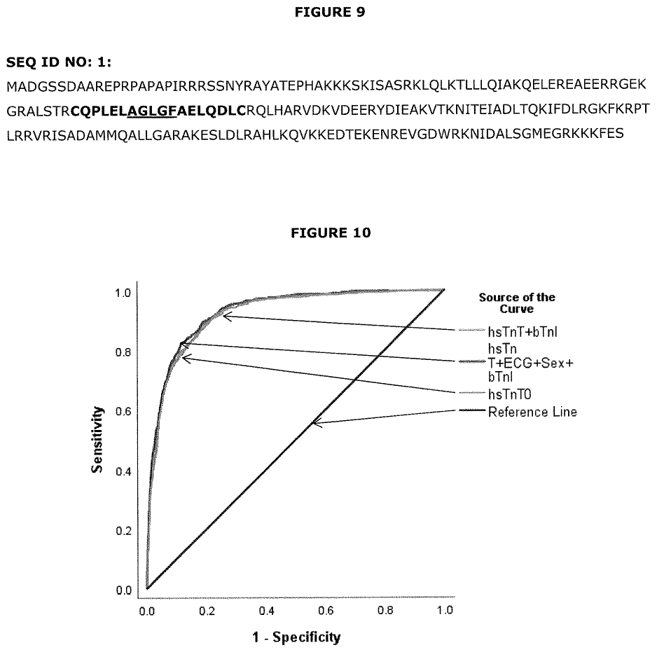

Immunoassays specific for bTnI-IgG usually will require the production or sourcing of antibodies that specifically bind to bTnI-IgG. The antibodies can be used to construct immunoassays with broad specificity, as in competitive binding assays below, or used in conjunction with other antibodies described below in sandwich type assays to produce assays specific to bTnI-IgG. The person skilled in the art will appreciate that non-competitive assays are also possible. The latter antibodies for sandwich immunoassays include those specific for amino acid sequences including the specific epitope sequences defined within SEQ ID NO:1 and illustrated in FIG. 9.

In another example, indicators may also be used. Indicators may be employed in ELISA and RIA assay formats.

Polyclonal and monoclonal antibodies can be used in competitive binding or sandwich type assays. In one example of this method a liquid sample is contacted with the antibody and simultaneously or sequentially contacted with a labelled bTnI-IgG or modified peptide containing the epitope recognised by the antibody.

The label can be a radioactive component such as ¹²⁵I, ¹³¹I, ³H, ¹⁴C or a non-radioactive component that can be measured by time resolved fluorescence, fluorescence, fluorescence polarisation, luminescence, chemiluminescence or colorimetric methods. These compounds include europium or other actinide elements, acrinidium esters, fluorescein, or radioactive material such as those above, that can be directly measured by radioactive counting, measuring luminescent or fluorescent light output, light absorbance etc. The label can also be any component that can be indirectly measured such as biotin, digoxin, or enzymes such as horseradish peroxidase, alkaline phosphatase. These labels can be indirectly measured in a multitude of ways. Horseradish peroxidase for example can be incubated with substrates such as o-Phenylenediamine Dihyhdrochloride (OPD) and peroxide to generate a coloured product whose absorbance can be measured, or with luminol and peroxide to give chemiluminescent light which can be measured in a luminometer. Biotin or digoxin can be reacted with binding agents that bind strongly to them; e.g. avidin will bind strongly to biotin. These binding agents can in turn be covalently bound or linked to measurable labels such as horseradish peroxidase or other directly or indirectly measured labels as above. These labels and those above may be attached to the peptide or protein: during synthesis, by direct reaction with the label, or through the use of commonly available crosslinking agents such as MCS and carbodiimide, or by addition of chelating agents.

Following contact with the antibody, usually for 18 to 25 hours at 4 °C, or 1 to 240 minutes at 30 °C to 40 °C, the labelled peptide bound to the binding agent (antibody) is separated from the unbound labelled peptide. In solution phase assays, the separation may be accomplished by addition of an anti-gamma globulin antibody (second-antibody) coupled to solid phase particles such as cellulose, or magnetic material. The second-antibody is raised in a different species to that used for the primary antibody and binds the primary antibody. All primary antibodies are therefore bound to the solid phase via the second antibody. This complex is removed from solution by centrifugation or magnetic attraction and the bound labelled peptide measured using the label bound to it. Other options for separating bound from free label include formation of immune complexes, which precipitate from solution, precipitation of the antibodies by polyethyleneglycol or binding free labelled peptide to charcoal and removal from solution by centrifugation of filtration. The label in the separated bound or free phase is measured by an appropriate method such as those presented above.

Competitive binding assays can also be configured as solid phase assays that are easier to perform and are therefore preferable to those above. This type of assay use a solid support including plates with wells (commonly known as ELISA or immunoassay plates), solid beads or the surfaces of tubes. The primary antibody is either adsorbed or covalently bound to the surface of the plate, bead or tube, or is bound indirectly through a second anti gamma globulin or anti Fc region antibody adsorbed or covalently bound to the plate. Sample and labelled peptide (as above) are added to the plate either together or sequentially and incubated under conditions allowing competition for antibody binding between bTnI-IgG in the sample and the labelled peptide. Unbound labelled peptide can subsequently be aspirated off and the plate rinsed leaving the antibody bound labelled peptide attached to the plate. The labelled peptide can then be measured using techniques described above.

Sandwich type assays are more preferred for reasons of specificity, speed and greater measuring range. In this type of assay an excess of the primary antibody to bTnI-IgG is attached to the well of an ELISA plate, bead or tube via adsorption, covalent coupling, or an anti Fc or gamma globulin antibody, as described above for solid phase competition binding assays. Sample fluid or extract is contacted with the antibody attached to the solid phase. Because the antibody is in excess this binding reaction is usually rapid. A second antibody to a bTnI-IgG peptide complex is also incubated with the sample either simultaneously or sequentially with the primary antibody. This second antibody is chosen to bind to a site on bTnI-IgG that is different from the binding site of the primary antibody. These two antibody reactions result in a sandwich with the bTnI-IgG from the sample sandwiched between the two antibodies. The second antibody is usually labelled with a readily measurable compound as detailed above for competitive binding assays. Alternatively, a labelled third antibody which binds specifically to the second antibody may be contacted with the sample. After washing the unbound material the bound labelled antibody can be measured by methods outlined for competitive binding assays. After washing away the unbound labelled antibody, the bound label can be quantified as outlined for competitive binding assays.

A dipstick type assay may also be used. These assays are well known in the art. They may for example, employ small particles such as gold or coloured latex particles with specific antibodies attached. The liquid sample to be measured may be added to one end of a membrane or paper strip preloaded with the particles and allowed to migrate along the strip. Binding of the antigen in the sample to the particles modifies the ability of the particles to bind to trapping sites, which contain binding agents for the particles such as antigens or antibodies, further along the strip. Accumulation of the coloured particles at these sites results in colour development are dependent on the concentration of competing antigen in the sample. Other dipstick methods may employ antibodies covalently bound to paper or membrane strips to trap antigen in the sample. Subsequent reactions employing second antibodies coupled to enzymes such as horse radish peroxidase and incubation with substrates to produce colour, fluorescent or chemiluminescent light output will enable quantitation of antigen in the sample.

### Receiver Operating Characteristic (ROC) Analysis

The clinical performance of a laboratory test depends on its diagnostic/prognostic accuracy, or the ability to correctly classify subjects into clinically relevant subgroups. Prognostic accuracy measures the test's ability to correctly distinguish two different conditions of the subjects investigated. Such conditions are for example health and disease or benign versus malignant disease.

In each case, a receiver operating characteristic (ROC) plot depicts the overlap between the two distributions by plotting the sensitivity versus 1-specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1-specificity [defined as (number of false-positive results)/(number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test.

One convenient objective to quantify the diagnostic accuracy of a laboratory test is to express its performance by a single number. The most common global measure is the area under the ROC plot. By convention, this area is always ≥0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

### Test Kits & Articles of Manufacture

Typically, test kits or articles of manufacture will be formatted for assays known in the art (e.g.) ELISA assays.

Binding agents that selectively bind cardiac troponins as well as immunoglobulins bound to cardiac troponins (e.g. IgG), and which include antigenic variants, thereof are desirably included in the test kits or articles of manufacture.

Accordingly, in an aspect of the present invention there is provided a test kit or article of manufacture comprising:
(i) an anti-cTnI binding agent which selectively binds to an equivalent region comprising amino acids 80-97 of cTnI as defined by SEQ ID NO: 1; and
(ii) an anti-human IgG binding agent which selectively binds to the IgG bound to cTnI.

In another aspect of the present invention there is provided a test kit or article of manufacture for:
(a) diagnosing an acute coronary syndrome in a patient including an acute myocardial infarction or unstable angina pectoris;
(b) enhancing the performance of cardiac troponin, including highly sensitive troponin T (hsTnT), for diagnosing myocardial infarction;
(c) distinguishing between Type 1 and Type 2 myocardial infarction;
(d) predicting a new acute coronary syndrome event;
(e) predicting future stroke;
(f) predicting mortality; and
(g) predicting a positive stress test outcome,
   the test kit or article of manufacture comprising:
(i) an anti-cTnI binding agent which selectively binds to an equivalent region comprising amino acids 80-97 of cTnI as defined by SEQ ID NO: 1;
(ii) an anti-human IgG binding agent which selectively binds to the IgG bound to cTnI; and
(iii) optionally instructions for diagnosing or predicting the conditions recited in any one of (a) to (e).

In an example according to this and other aspects of the present invention, the binding agent is selected from an antibody or antigen-binding fragment and an aptamer.

In another example, the antibody is monoclonal antibody, for example, and may be prepared in any mammal as described above, and includes antigen binding fragments and antibodies prepared using native and fusion peptides, for example.

Accordingly, in yet another aspect of the present invention there is provided a test kit or article of manufacture for:
(a) diagnosing an acute coronary syndrome in a patient including an acute myocardial infarction or unstable angina pectoris;
(b) enhancing the performance of cardiac troponin, including highly sensitive troponin T (hsTnT), for diagnosing myocardial infarction;
(c) predicting a new acute coronary syndrome event;
(d) predicting future stroke; and
(e) predicting mortality
   the test kit or article of manufacture comprising:
(i) an anti-cTnI monoclonal antibody which selectively binds to an equivalent region comprising amino acids 80-97 of cTnI as defined by SEQ ID NO: 1;
(ii) an anti-human IgG monoclonal antibody which selectively binds to the IgG bound to cTnI; and
(iii) optionally instructions for diagnosing or predicting the conditions recited in any one of (a) to (e).

The test kits or articles of manufacture may be comprised of one or more containers and may also include collection equipment, for example, bottles, bags (such as intravenous fluids bags), vials, syringes, and test tubes. At least one container will be included and will hold a product which is effective for use in the assays and methods described herein. The product is typically a peptide binding agent, particularly an antibody or antigen-binding fragment according to the invention described herein, or a composition comprising any of these. In one example, an instruction or label on or associated with the container indicates that the composition is used for diagnosing or monitoring the acute coronary syndrome or for predicting a future acute coronary syndrome event, stroke or mortality. Other components may include diluents and buffers.

The test kits or articles of manufacture may also include detection or measurement means or measurement directions involving one or more additional markers or risk factors for a cardiac disease of interest (e.g.) heart rate, haemoglobin concentration, blood pressure, age, sex, weight, level of physical activity, family history of events including obesity, diabetes and cardiac events, and levels of NT-proBNP, BNP, BNPsp and BNPsp fragments including BNPsp(17-26). Again, this may include binding agent(s), aptamer(s), antibody(s) and antigen-binding fragment(s) thereof which selectively bind to other biomarker(s) of interest.

In certain examples of the test kits or articles of manufacture according to the present invention, the anti-cTnI monoclonal antibody which selectively binds to an equivalent region comprising amino acids 80-97 of cTnI as defined by SEQ ID NO: 1 is immobilized on a solid substrate, surface or matrix, for example, a micro-titre plate, porous strip or chip to form at least one detection site for the bTnI-IgG complex.

Also included in the kits or articles of manufacture may be a device for sample analysis comprising a disposable testing cartridge with appropriate components (markers, antibodies and reagents) to carry out sample testing. The device will conveniently include a testing zone and test result window. Immunochromatographic cartridges are examples of such devices. See for example U.S. Pat. Nos. 6,399,398; 6,235,241 and 5,504,013.

Alternatively, the device may be an electronic device which allows input, storage and evaluation of levels of the measured marker against control levels and other marker levels. US 2006/0234315 provides examples of such devices. Also useful in the invention are Ciphergen's Protein Chip® which can be used to process SELDI results using Ciphergen's Protein Chip® software package.

The invention is further described with reference to the following examples. It will be appreciated that the invention as claimed is not intended to be limited in any way by these examples.

### Examples

### Example 1: Assay Method

A monoclonal antibody directed to amino acids 86-90 of the troponin I protein was coated on an ELISA plate. The plate is washed and then blocked with a common blocking agent (e.g. bovine serum albumin, casein) resulting in a prepared test plate.

Test plates are then processed as follows.

100 ul of plasma sample (diluted 1:200 in PBS) is added to the plate wells and incubated at room temperature for 1 hour. Wells are washed with 300 ul of buffer (0.1% Tween?) aspirated dry. 100 ul of goat anti-human IgG antibody conjugated to HRP is added and incubated at room temperature for 30 minutes. Plates are then washed again, aspirated dry and 100 ul of TMB added to develop colour.

The assay is therefore a sandwich ELISA setup that produces a linear curve proportional to the amount of antibody bound TnI present. Crucial to the assay is the requirement that antibody bound TnI must be free of bound TnT: the plate bound monoclonal antibody is directed to the region of TnI that binds to TnT—bound TnT will interfere with this reaction and thus TnI cannot bind if TnI and TnT are complexed. Refer to FIG. 1.

### Example 2: Clinical Samples Tested

### Study Population and Design

Patients with chest pain suspicious of acute coronary syndromes (ACS) were prospectively enrolled into the Applicant's ongoing observational study known as Signal Peptides in Acute Coronary Events (SPACE, http://www.anzctr.org.au, ACTRN12609000057280). All patients were enrolled in accord with protocols approved by the Health and Disabilities Ethics Committee of the Ministry of Health, New Zealand. All participants gave informed consent before recruitment and all investigations conformed to the principles of the Declaration of Helsinki. Since March 2009, 1053 eligible patients aged 18 years or older with the primary complaint of acute chest pain clinically suspicious of ACS and 4 hours from onset were recruited and included in this study. More general/atypical symptoms (such as fatigue, nausea, vomiting, sweating and faintness) were not used as inclusion criteria. Patients with end stage renal disease on dialysis were excluded.

Subsequent to this, and to further validate the initial findings/correlations discovered by the present applicants, patient samples were obtained and tested from the FAST-TRAC and APACE patient cohorts and are described in the examples which follow. Together with SPACE, and collectively referred to herein, as expanded patient cohorts for the measurement of bTnI-IgG and other clinical variables.

### Adjudicated Diagnosis

The adjudicated diagnosis of acute MI was made in accordance with published guidelines [8], by two independent cardiologists with access to all clinical data, but not BNP signal peptide or hsTnT assay results. In the case of disagreement, an independent third cardiologist adjudicated to resolve this. The biochemical component of the diagnosis of MI was based on a highly sensitive (hs) TnI assay with 1 value≥99th URL (Abbott Architect, unisex cut-off of 28 ng/L) within 12 hours of presentation. Atrial fibrillation (AF) during emergency department presentation was determined from the ECG, whereas the diagnosis of UAP was made on the basis of confirmatory provocative investigations (exercise tolerance testing (ETT) or dobutamine stress echocardiography testing (DSE)) or angiographic catheterisation findings. Other cardiac disorders were defined as non-ACS cardiac presentations comprising conduction disorders (sick sinus syndrome), arrhythmias (atrial fibrillation/flutter) and acute heart failure. Undifferentiated chest pain was defined as chest pain without definitive associated clinical findings or cardiac tests where doubt remains as to the aetiology. Non-cardiac chest pain was defined as present when a definite non-cardiac cause for symptoms was identified.

### Follow-Up and Prognostic End Points

Within 730 days post-discharge, patients were followed up by telephone or in writing. Reported clinical events were identified from the patients themselves (or their primary physician) corroborated by the records of the treating institution or by the centralised New Zealand Ministry of Health database registry entries on mortality and events. The post-discharge end points considered were death, new MI, new ACS, acute decompensated heart failure and stroke. Events were analysed by ROC analysis for three groups; all patients (n=1053), MI patients (n=217) and non-MI patients (n=836).

### Clinical Assessment and Sample Collection

For all patients, initial assessment included clinical history, physical examination, ECG recordings, standard blood tests, pulse oximetry and chest radiography. Patient management was at the discretion of the attending physicians. Only standard clinical core lab hsTnI (Abbott Architect) and other standard blood test results were available to treating staff.

After consent was given, serial blood samples for measurement of bTnI-IgG, hsTnT and NT-proBNP (EDTA tubes) and hsTnI and lipids (Heparin tubes) were taken at 0, 1, 2 and 12-24 hours after presentation. Blood samples (10 mL) were drawn into EDTA tubes chilled on ice, centrifuged at 2500 g for 10 minutes and the plasma frozen at −80 °C prior to assays. Heparin samples were collected into 5 ml tubes and immediately sent to the hospital core biochemistry unit for measurement of hsTnI and lipids.

### Statistical Analysis

Continuous variables are presented as median (interquartile range, (IQR)) and categorical variables as numbers and percentages. Bivariate associations between patient outcomes and continuous variables were analysed using non-parametric Mann-Whitney U test and categorical variables using the Pearson χ² test. Analysis of plasma analyte results employed Spearman rank order correlation testing and receiver operator characteristic curve (ROC) analysis and diagnostic performance (sensitivity, specificity, positive predictive value (PPV) and negative predictive values (NPV)) were carried out using SPSS v28 (IBM). For ROC curve generation and biomarker panel comparisons, biomarker data were analysed as standardised variables (z-scores) or Log10 values, where appropriate.

Individual biomarkers (bTnI-IgG, NT-proBNP, hsTnI and hsTnT) were assessed by ROC analysis for the prediction of index MI and UAP.

ROC curve comparisons were made using the approach of Hanley and McNeill [9] in SPSS v28. In all analyses, a p-value<0.05 was considered significant.

### Example 3: Quantification of bTnI-IgG Levels in Space Cohort

The median (interquartile range, 25^{th}-75^{th} percentile) values of bTnI-IgG in individuals with different diagnoses are reflected in FIG. 2. NCCP=non-cardiac chest pain, Possible UAP=possible unstable angina, Other Cardiac=other cardiac disorder diagnoses (e.g. heart failure, rhythm disorders), definite UAP=definite unstable angina, NSTEMI=non-ST elevated myocardial infraction, STEMI=ST-elevated myocardial infarction. All ACS related disorders (definite UA, NSTEMI, STEMI) had lower bTnI-IgG levels compared non-ACS diagnoses.

These data show median interquartile ranges of bTnI-IgG were lower in patients with an adjudicated diagnosis of an acute coronary syndrome (e.g.) STEMI, NSTEMI and UAP, as compared to patients with an adjudicated diagnosis of non-acute (e.g. “other cardiac issues”) or non-cardiac issues (“NCCP”).

### Example 4: bTnI-IgG Levels Enhance Dx Performance of hsTnT

The data presented in FIG. 3A and Table 2 below reflects diagnostic receiver operator characteristic (ROC) curves for the diagnosis of acute myocardial infarction (AMI) (n=217) by hsTnI (blue line), hsTnT (green line) and bTnI-IgG (orange line) in 1053 patients presenting to hospital (“index presentation”) with the primary complaint of chest pain suspicious of an acute coronary syndrome.

TABLE 2
bTnI-IgG assisted performance of hsTnT
for Dx of MI (all bTnI-IgG conc.)

| | AUC | Sig | Lower CI | Upper CI | p |
|---|---|---|---|---|---|
| bTnI-IgG | 0.442 | 0.004 | 0.402 | 0.481 | |
| hsTnI0 | 0.954 | <0.001 | 0.938 | 0.970 | |
| hsTnT0 | 0.925 | <0.001 | 0.903 | 0.946 | 0.004 v hsTnI |

The data presented in FIG. 3B reflects the diagnostic receiver operator characteristic (ROC) curves for the diagnosis of acute myocardial infarction (AMI) (n=86) by hsTnI (blue line), hsTnT (green line) and bTnI-IgG (orange line) in 474 patients presenting to hospital with the primary complaint of chest suspicious of ACS. In all patients, the value of bTnI-IgG was above the entire group (n=1053) median of 0.9659.

The data presented in FIG. 3C and Table 3 below reflects the diagnostic receiver operator characteristic (ROC) curves for the diagnosis of acute myocardial infarction (AMI) (n=131) by hsTnI (blue line), hsTnT (green line) and bTnI-IgG (orange line) in 515 patients presenting to hospital with the primary complaint of chest suspicious of ACS. In all patients, the value of bTnI-IgG was lower than the entire group (n=1053) median of 0.9659.

TABLE 3
bTnI-IgG assisted performance of hsTnT
for Dx of MI (bTnI-IgG conc < median)

| | AUC | Sensitivity | Specificity | NPV | PPV |
|---|---|---|---|---|---|
| hsTnI0 | 0.955 | 98.8 | 49.5 | 99.5 | 30.2 |
| hsTnT0 | 0.958 | 98.8 | 68.6 | 99.6 | 41.1 |

Accordingly, the performance of hsTnT improves from an AUC of 0.925 (FIG. 3A, Table 2) to an AUC of 0.958 (FIG. 3C, Table 3) when bTnI-IgG levels below the median value for the entire cohort are factored into the model.

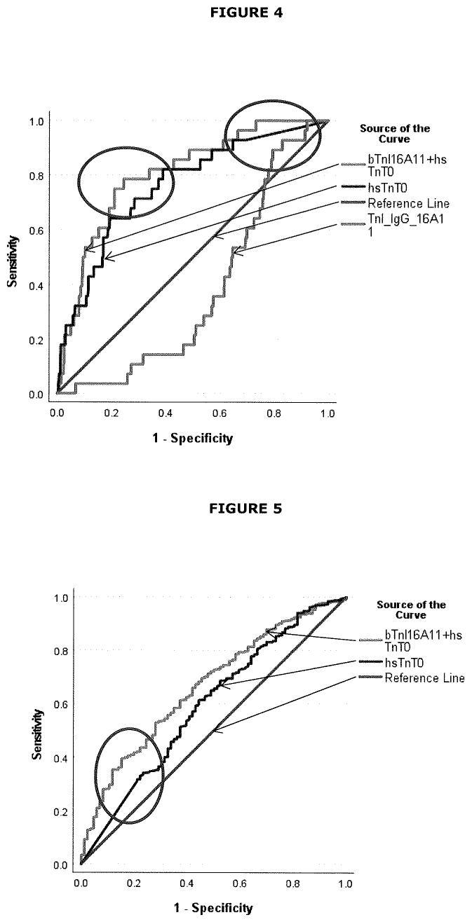

These observations are further validated by the data presented in Table 4 below and read in conjunction with FIG. 4, where bTnI-IgG measurements added to hsTnT, when hsTnT levels are lower than the 99th percentile of 15 ng/L (blue line), significantly improve the ROC diagnostic performance of hsTnT alone for the diagnosis of acute myocardial infarction (n=28, green line) (p=0.044). bTnI-IgG levels alone (orange line) are lower in those with AMI compared with non-AMI diagnoses (n=655, P=0.002).

TABLE 4
bTnI-IgG assisted performance of hsTnT for
Dx of MI [“rule in test”]

| | ROC AUC | Sensitivity | Specificity | NPV | PPV | p |
|---|---|---|---|---|---|---|
| hsTnT | 0.76 | 100 | 0 | — | 4.0 | |
| hsTnT + | 0.81 | 100 | 27 | 100 | 5.4 | 0.049 |
| bTnI-IgG | | | | | | |
| hsTnT | | 79 | 63 | 98.6 | 8.2 | |
| hsTnT + | | 79 | 76 | 98.9 | 12.1 | 0.049 |
| bTnI-IgG | | | | | | |

According to the data presented in FIG. 5 presented in conjunction with Table 5 below, bTnI-IgG measurements added to hsTnT, when hsTnT levels are lower than the 99th percentile of 15 ng/L (green line), significantly improve the ROC diagnostic performance of hsTnT alone for the rule out of acute myocardial infarction (n=597 out of 683 total cases, blue line). The positive predictive value of hsTnT for rule out of AMI increased from 89% to 95.1% (P<0.001).

TABLE 5
bTnI-IgG assisted performance of hsTnT for
Dx of MI [“rule out test”]

| | ROC AUC | Sensitivity | Specificity | NPV | PPV | p |
|---|---|---|---|---|---|---|
| hsTnT | 0.60 | 12 | 90 | 12.8 | 89.3 | |
| hsTnT + | 0.67 | 28 | 90 | 15.3 | 95.1 | <0.001 |
| bTnI-IgG | | | | | | |

### Example 5: bTnI-IgG Levels Fail to Enhance DX Performance of hsTnI

Addition of bTnI-IgG to hsTnI measurements for the diagnosis of acute myocardial infarction made no change to the receiver operating curve performance of hsTnI (FIGS. 6A and 6B read in conjunction with Tables 6A and 6B, below). The ROC curve of hsTnI did not change in the whole group of n=1053 (FIG. 6A) or in those with a hsTnI less than 30 ng/L (FIG. 6B).

The data accompanying FIG. 6A is as follows:

TABLE 6A
bTnI-IgG assisted performance of hsTnI for Dx of MI when (all hsTnI conc.)

| | | | | Asymptotic 95% Confidence | |
|---|---|---|---|---|---|
| Test Result | | Std. | Asymptotic | Interval | |
| Variable(s) | Area | Error^{a} | Sig.^{b} | Lower Bound | Upper Bound |
| TnI_IgG_16A11 | 0.441 | 0.020 | 0.003 | 0.402 | 0.480 |
| hsTnI0 | 0.955 | 0.008 | 0.000 | 0.940 | 0.970 |
| bTn16A11 + hsTnI0 | 0.955 | 0.007 | 0.000 | 0.941 | 0.970 |

The data accompanying FIG. 6B is as follows:

TABLE 6B
bTnI-IgG assisted performance of hsTnI for Dx of MI (hsTnI ≤ 30 ng/L)

| | | | | Asymptotic 95% Confidence | |
|---|---|---|---|---|---|
| Test Result | | Std. | Asymptotic | Interval | |
| Variable(s) | Area | Error^{a} | Sig.^{b} | Lower Bound | Upper Bound |
| TnI_IgG_16A11 | 0.422 | 0.034 | 0.020 | 0.356 | 0.488 |
| hsTnI0 | 0.888 | 0.020 | 0.000 | 0.848 | 0.927 |
| bTn16A11 + hsTnI0 | 0.889 | 0.020 | 0.000 | 0.850 | 0.928 |

These data reflect that bTnI-IgG failed to add to the performance of hsTnI for the diagnosis of myocardial infarction, further validating the assay approach designed by the Applicant where bTnI-IgG indirectly reflects the amount of cTnT bound to TnI in TnI-TnT interactions.

### Example 6: bTnI-IgG To Predict Future ACS

The utility of bTnI-IgG was next examined for its ability to enhance the performance of cTnT to predict a future acute coronary syndrome event. The results of these analyses presented in Table 7 below and reflect that assay specificity was enhanced by the presence of bTnI-IgG, particularly for new ACS (including UAP) within two years and mortality within four years (i.e. from index presentation).

TABLE 7
bTnI-IgG assisted Px outcomes by hsTnT

| | | ROC | Sensitivity % | Specificity % | PPV % | NPV % |
|---|---|---|---|---|---|---|
| Death 4 yr | hsTnT0 | 0.710 | 90 | 29 | 12.2 | 96.3 |
| (n = 98) | hsTnT0 + bTnI- | 0.740** | 90 | 42 | 14.6 | 97.4 |
| | IgG | | | | | |
| New MI 2 yr | hsTnT0 | 0.705 | 90 | 32 | 10.2 | 97.4 |
| (n = 78) | hsTnT0 + bTnI- | 0.720# | 90 | 32 | 10.2 | 97.4 |
| | IgG | | | | | |
| New UAP 2 yr | hsTnT0 | 0.595 | 90 | 24 | 6.9 | 97.5 |
| (n = 58) | hsTnT0 + bTnI- | 0.636** | 90 | 29 | 7.3 | 97.9 |
| | IgG | | | | | |
| New ACS 2 yr | hsTnT0 | 0.651 | 90 | 25 | 13.5 | 95.0 |
| (n = 114) | hsTnT0 + bTnI- | 0.681** | 90 | 30 | 14.3 | 95.8 |
| | IgG | | | | | |

**p < 0.001 v hsTnT;
#p = 0.065 v hsTnT

Data generated subsequently (e.g. refer to Example 13 below) further demonstrates a utility for bTnI-IgG to predict a new ACS within ~1 year or <365 days.

### Example 7: bTnI-IgG To Predict Future Stroke

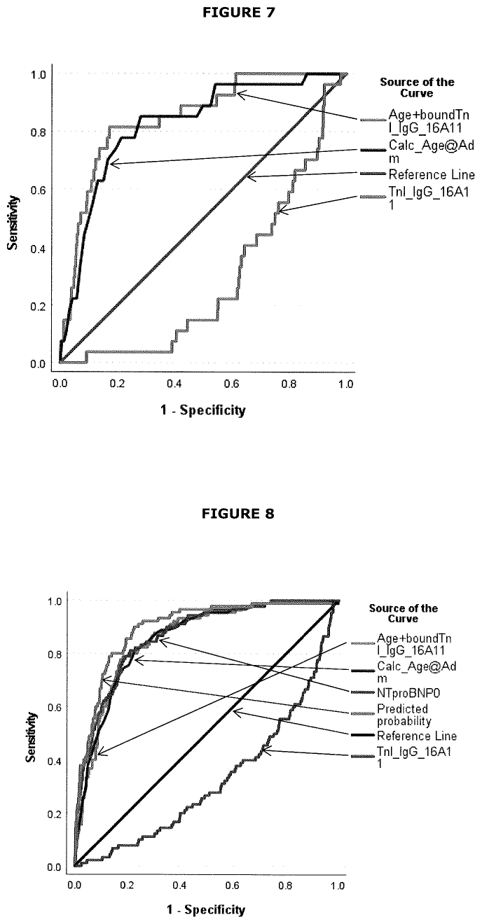

The data presented in FIG. 7/Tables 8 and 9 below reflects that lower bTnI-IgG values predict stroke within two years (n=27) in those who have previously presented to hospital with chest pain (n=1053). Further, bTnI-IgG added to the most powerful predictor of stoke events (age) and doubled its specificity, outperforming a currently clinically indicated marker of new cardiovascular events (i.e.) NT-proBNP.

TABLE 8
ROC performance of TnI-IgG for Px of stroke within two years

| | | | | Asymptotic 95% Confidence | |
|---|---|---|---|---|---|
| | | Std. | Asymptotic | Interval | |
| Test Result Variable(s) | Area | Error^{a} | Sig.^{b} | Lower Bound | Upper Bound |
| TnI_IgG_16A11 | 0.289 | 0.041 | 0.000 | 0.209 | 0.370 |
| Calc_Age@Adm | 0.826 | 0.039 | 0.000 | 0.750 | 0.902 |
| Age + boundTnI_IgG_16A11 | 0.850 | 0.035 | 0.000 | 0.780 | 0.919 |

TABLE 9
Test variables in the equation

| | | | | | | | | 95% C.I for | |
|---|---|---|---|---|---|---|---|---|---|
| | | | | | | | | EXP(B) | |
| | | B | S.E. | Wald | df | Sig. | Exp(B) | Lower | Upper |
| Step 1^{a} | Zscore(Calc_Age@Adm) | 1.337 | 0.282 | 22.543 | 1 | <0.001 | 3.806 | 2.192 | 6.608 |
| | Zscore(hsTnI0) | 0.224 | 0.129 | 3.023 | 1 | 0.082 | 1.251 | 0.972 | 1.611 |
| | Zscore(TnI_IgG_16A11) | −0.845 | 0.347 | 5.931 | 1 | 0.015 | 0.430 | 0.218 | 0.848 |
| | Constant | −4.822 | 0.411 | 137.725 | 1 | <0.001 | 0.008 | | |

### Example 8: bTnI-IgG To Predict Future Mortality

The data presented in FIG. 8/Table 7 above and Tables 10 and 11 below reflects that bTnI-IgG is an independent predictor of mortality within 4 years (n=90) in chest pain patients (n=1053, both ACS and non-ACS). When combined with age and NT-proBNP, bTnI-IgG forms an important and significant component of the combined model to predict such mortality where the AUC=0.896±0.017 (orange line in FIG. 8).

TABLE 10
ROC performance of TnI-IgG for Px of mortality within four years

| | | | | Asymptotic 95% Confidence | |
|---|---|---|---|---|---|
| | | Std. | Asymptotic | Interval | |
| Test Result Variable(s) | Area | Error^{a} | Sig.^{b} | Lower Bound | Upper Bound |
| TnI_IgG_16A11 | 0.322 | 0.030 | 0.000 | 0.264 | 0.380 |
| Calc_Age@Adm | 0.854 | 0.020 | 0.000 | 0.816 | 0.893 |
| NTproBNP0 | 0.866 | 0.018 | 0.000 | 0.830 | 0.902 |
| Age + boundTnI_IgG_16A11 | 0.857 | 0.019 | 0.000 | 0.819 | 0.895 |
| Whole model | 0.896 | 0.017 | 0.000 | 0.863 | 0.929 |

TABLE 11
Test variables in the equation

| | | | | | | | | 95% C.I for | |
|---|---|---|---|---|---|---|---|---|---|
| | | | | | | | | EXP(B) | |
| | | B | S.E. | Wald | df | Sig. | Exp(B) | Lower | Upper |
| Step 1^{a} | Zscore(TnI_IgG_16A11) | −0.454 | 0.176 | 6.640 | 1 | 0.010 | 0.635 | 0.450 | 0.897 |
| | Zscore(Calc_Age@Adm) | 1.099 | 0.200 | 30.189 | 1 | <0.001 | 3.000 | 2.027 | 4.439 |
| | AngHx | 0.926 | 0.320 | 8.367 | 1 | 0.004 | 2.525 | 1.348 | 4.731 |
| | eGFR_CKDepi | −0.024 | 0.010 | 5.692 | 1 | 0.017 | 0.977 | 0.958 | 0.996 |
| | Zscore(NTproBNP0) | 0.445 | 0.108 | 16.952 | 1 | <0.001 | 1.561 | 1.263 | 1.929 |
| | Constant | −2.617 | 0.665 | 15.468 | 1 | <0.001 | 0.073 | | |

The additional examples which follow demonstrates:
1. bTnI-IgG improves hsTnT based diagnosis of MI, especially when hsTnT at presentation is below cut-off of <15 ng/L (e.g. <14 ng/L) in a significantly expanded patient cohort including SPACE, FAST-TRAC and APACE (Examples 9A and 9B; further reinforces the data presented in Example 4);
2. bTnI-IgG at index presentation, and also a delta change of bTnI-IgG between 0-2 hrs, can detect patients who have Type 2 myocardial infarction (Examples 10 and 12);
3. incorporation of bTnI-IgG performance from (2) into pathways and random forest statistical analyses can improve the positive predictive value of Troponin T to detect Type 1 myocardial infarction (Example 11);
4. bTnI-IgG can assist prediction of a new ACS event within 365 day in a patient who presented to ED and did NOT receive and index diagnosis of MI (Example 13);
5. bTnI-IgG can also predict new ACS within 365 days in patients who had good evidence of significant CAD (either T2 MI, UAP or radiology findings of 70% occlusion) (Example 13);
6. bTnI-IgG can also do the same in 5) in patients who did NOT have an index diagnosis of MI (Example 13);
7. bTnI-IgG improves the pre-test probability of a positive cardiac stress test in combination with GDF-15 measurement (Example 14); and
8. bTnI-IgG improves the pre-test probability of a positive cardiac stress test in combination with GDF-15 measurement and troponin I (Example 14).

### Example 9A: Diagnosis of Myocardial Infarction in an Expanded Patient Cohort (all Concentrations)

The data presented in FIG. 10 read in conjunction with Tables 12-13 below reflects the ROC performance of bTnI-IgG in enhancing the diagnosis of an acute myocardial infarction by hsTnT in an expanded patient cohort comprising SPACE, FAST-TRAC and APACE.

TABLE 12
ROC performance of hsTnT for Dx of MI in the presence of bTnI-IgG,
sex and abnormal ECG in expanded patient cohort (n = 678/4276)

| | | | | Asymptotic 95% Confidence | |
|---|---|---|---|---|---|
| | | Std. | P | Interval | |
| Test Result Variable(s) | Area | Error^{a} | value | Lower Bound | Upper Bound |
| hsTnT | 0.920 | 0.005 | | 0.909 | 0.930 |
| hsTnT + bTnI-IgG | 0.922 | 0.005 | 0.021 | 0.912 | 0.933 |
| hsTnT + bTnI-IgG + sex + | 0.929 | 0.005 | 0.001 | 0.919 | 0.939 |
| ECG | | | | | |

TABLE 13
Test variables in the equation

| | | | | | | | | 95% C.I for | |
|---|---|---|---|---|---|---|---|---|---|
| | | | | | | | | EXP(B) | |
| | | B | S.E. | Wald | df | Sig. | Exp(B) | Lower | Upper |
| Step 1^{a} | Log10_hsTnT0 | 3.670 | 0.139 | 694.377 | 1 | <0.001 | 39.259 | 29.880 | 51.582 |
| | ECGSTD | 1.170 | 0.183 | 87.578 | 1 | <0.001 | 5.531 | 3.866 | 7.913 |
| | Sex_Male | 0.408 | 0.128 | 10.107 | 1 | 0.001 | 1.504 | 1.169 | 1.933 |
| | Zscore(bTnI-IgG) | −0.283 | 0.062 | 20.868 | 1 | <0.001 | 0.754 | 0.667 | 0.851 |
| | Constant | −6.840 | 0.226 | 915.104 | 1 | <0.001 | 0.001 | | |

### Example 9B3: Diagnosis of Myocardial Infarction in an Expanded Patient Cohort where hsTnT<14 ng/L

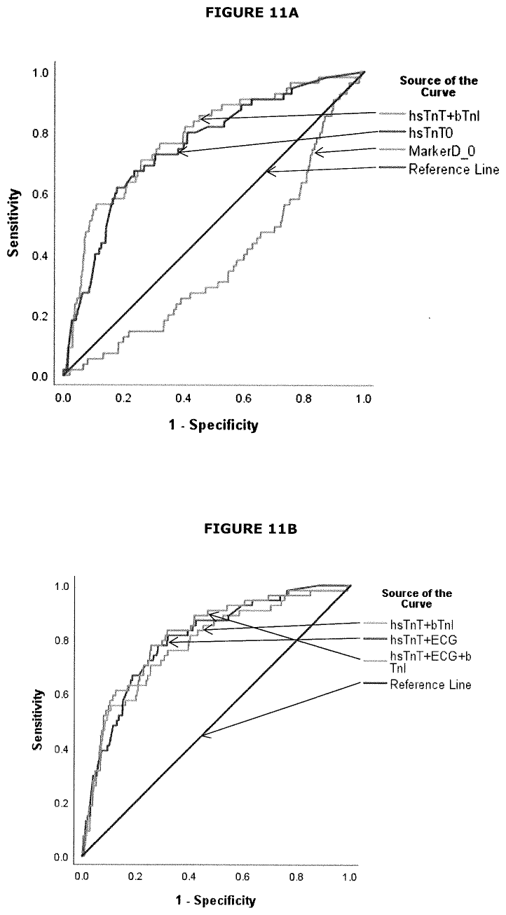

The data presented in FIGS. 11A and 11B read in conjunction with and Tables 14-15 below reflects the ROC performance of bTnI-IgG in enhancing the diagnosis of an acute myocardial infarction by hsTnT in an expanded patient cohort comprising SPACE, FAST-TRAC and APACE when the concentration of hsTnT at index presentation is <14 ng/L.

TABLE 14
ROC performance of hsTnT for Dx of MI in the presence
of bTnI-IgG when hsTnT < 14 ng/L at index presentation

| | AUC | Sens. | Spec. | NPV | PPV | P value |
|---|---|---|---|---|---|---|
| bTnI-IgG | 0.379 | | | | | |
| hsTnT | 0.757 | 29.0 | 92.0 | 98.5 | 6.6 | |
| hsTnT + | 0.783 | 51.0 | 92.0 | 99.0 | 11.1 | 0.043 |
| bTnI-IgG | | | | | | |

TABLE 15
ROC performance of hsTnT for Dx of MI in the presence
of bTnI-IgG and abnormal ECG when hsTnT < 14 ng/L
at index presentation (floating and fixed sensitivity)

| | AUC | Sens. | Spec. | NPV | PPV | P value |
|---|---|---|---|---|---|---|
| hsTnT + | 0.783 | 51.0 | 92.0 | 99.0 | 11.1 | 0.043 v |
| bTnI-IgG | | | | | | hsTnT |
| hsTnT + | 0.801 | 39.0 | 92.0 | 98.7 | 8.7 | 0.408 v |
| ECG | | | | | | hsTnT + |
| | | | | | | bTnI-IgG |
| hsTnT + | 0.815 | 52.0 | 92.0 | 99.0 | 11.3 | 0.056 v |
| ECG + | | | | | | hsTnT + |
| bTnI-IgG | | | | | | ECG |
| hsTnT + | | 95.0 | 25.0 | 99.6 | 2.4 | |
| bTnI-IgG | | | | | | |
| hsTnT + | | 95.0 | 26.0 | 99.6 | 2.5 | |
| ECG | | | | | | |
| hsTnT + | | 95.0 | 31.0 | 99.7 | 2.6 | |
| ECG + | | | | | | |
| bTnI-IgG | | | | | | |

### Example 10: bTnI-IgG Utility to Identify Type 2 Myocardial Infarction

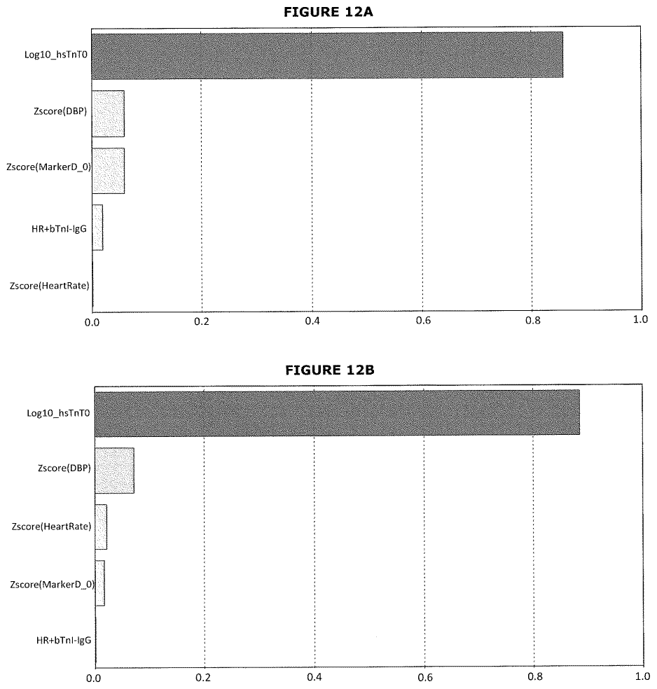

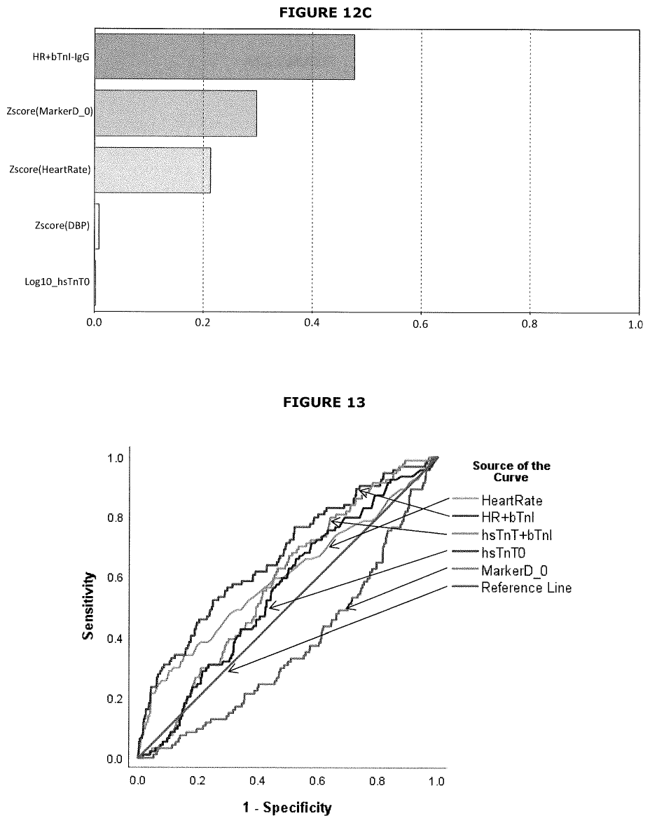

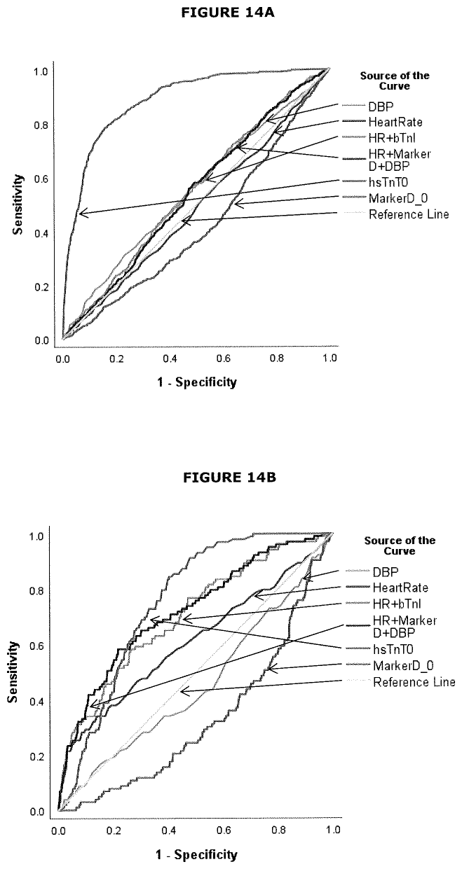

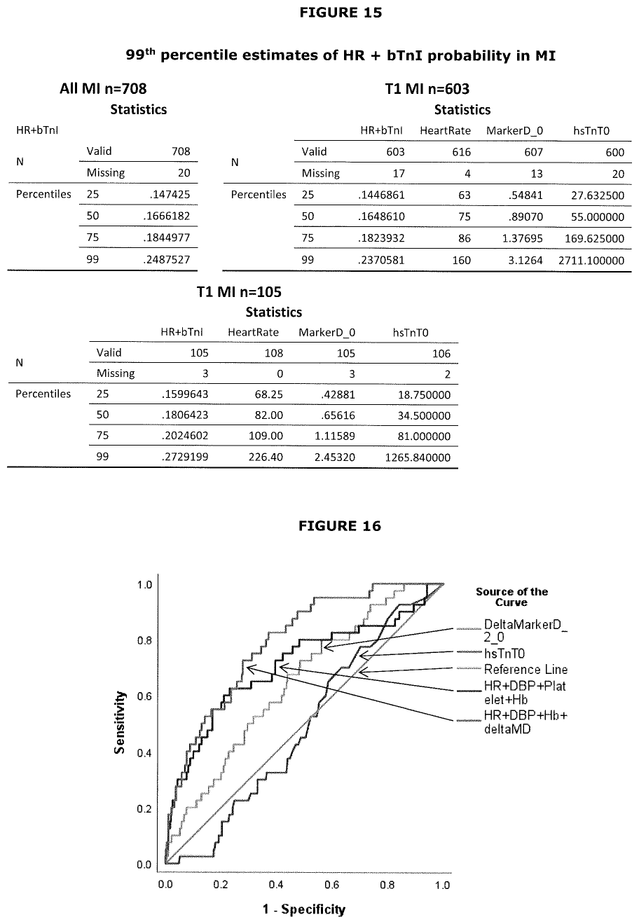

The data presented in FIGS. 12 and 13, read in conjunction with Tables 16-18 below, demonstrate that bTnI-IgG may be used to correctly identify patients with Type 2 myocardial infarction where hsTnT is >14 ng/L based on measured levels of bTnI-IgG at index presentation.

TABLE 16
Test variables in the equation

| | | | | | | | | 95% C.I for | |
|---|---|---|---|---|---|---|---|---|---|
| | | | | | | | | EXP(B) | |
| | | B | S.E. | Wald | df | Sig. | Exp(B) | Lower | Upper |
| Step 1^{a} | Zscore(bTnI-IgG) | −0.609 | 0.150 | 16.484 | 1 | <0.001 | 0.544 | 0.405 | 0.730 |
| | Zscore(heart rate) | 0.386 | 0.071 | 29.464 | 1 | <0.001 | 1.471 | 1.280 | 1.691 |
| | Constant | −2.994 | 0.135 | 492.092 | 1 | <0.001 | 0.050 | | |

TABLE 17
ROC performance of bTnI-IgG for Dx of Type 2 Myocardial Infarction

| | | | | Asymptotic 95% Confidence | |
|---|---|---|---|---|---|
| | | Std. | Asymptotic | Interval | |
| Test Result Variable(s) | Area | Error^{a} | Sig.^{b} | Lower Bound | Upper Bound |
| hsTnT0 | 0.556 | 0.029 | 0.051 | 0.500 | 0.612 |
| hsTnT0 + bTnI-IgG* | 0.581 | 0.027 | 0.003 | 0.528 | 0.633 |
| heart rate + bTnI-IgG** | 0.671 | 0.030 | 0.000 | 0.612 | 0.730 |
| heart rate | 0.596 | 0.034 | 0.005 | 0.529 | 0.663 |
| bTnI-IgG | 0.369 | 0.028 | 0.000 | 0.313 | 0.425 |

*P = 0.008
**P = 0.001

TABLE 18
Further Characteristics of ROC performance of
TnI-IgG for Dx of Type 2 Myocardial Infarction

| | | | P value | | |
|---|---|---|---|---|---|
| | | | bTnI-IgG v | | |
| | Sens. | | alternate | | |
| | (95% Spec.) | AUC | variable | NPV | PPV |
| hsTnT | 2.1 | 0.556 | | 93.5 | 2.7 |
| Heart rate | 21.3 | 0.596 | | 94.7 | 22.2 |
| bTnI-IgG | 10.6 | 0.369 | | 94.1 | 12.4 |
| hsTnT + | 3.2 | 0.581 | 0.008 | 93.6 | 4.1 |
| bTnI-IgG | | | | | |
| Heart rate + | 23.4 | 0.671 | 0.001 | 94.9 | 23.9 |
| bTnI-IgG | | | | | |

Further, the data presented in FIG. 14B shows that bTnI-IgG may be used to successfully identify Type 2 MI (n=103/3026) when hsTnT at index presentation is >5 ng/L (i.e. at a measurable level of Troponin T).

### Example 11: bTnI-IgG Utility to Identify Type 1 Myocardial Infarction

The data presented in FIG. 14A, read in conjunction with Tables 19-21 below, demonstrate the predictive power of hsTnT in the presence of bTnI-IgG>1.4 ng/L.

TABLE 19
PPV of hsTnT + bTnI-IgG for T1 MI when bTnI-IgG is >1.4 ng/L

| | | hsTnT AUC | | | |
|---|---|---|---|---|---|
| | Total n | for T1 MI | N MI | NPV | PPV |
| hsTnT | 4404 | 0.914 | 706 | | 84.8 |
| +bTnI-IgG > | 1264 | 0.922 | 138 | | 91.9 |
| 1.4 ng/L | | | | | |

TABLE 20
PPV of hsTnT + bTnI-IgG for T1 MI when hsTnT is >5 ng/L

| | | hsTnT AUC | | | |
|---|---|---|---|---|---|
| | Total n | for T1 MI | N MI | NPV | PPV |
| hsTnT | 3080 | 0.885 | 591 | | 85.0 |
| +bTnI-IgG > | 984 | 0.904 | 137 | | 92.0 |
| 1.4 ng/L | | | | | |

TABLE 21
PPV of hsTnT + bTnI-IgG for T1 MI when hsTnT is >14 ng/L

| | | hsTnT AUC | | | |
|---|---|---|---|---|---|
| | Total n | for T1 MI | N MI | NPV | PPV |
| hsTnT | 1482 | 0.781 | 552 | | 85.4 |
| +bTnI-IgG > | 401 | 0.767 | 130 | | 91.5 |
| 1.4 ng/L | | | | | |

### Example 12: ΔbTnI-IgG Utility to Identify Type 2 Myocardial Infarction

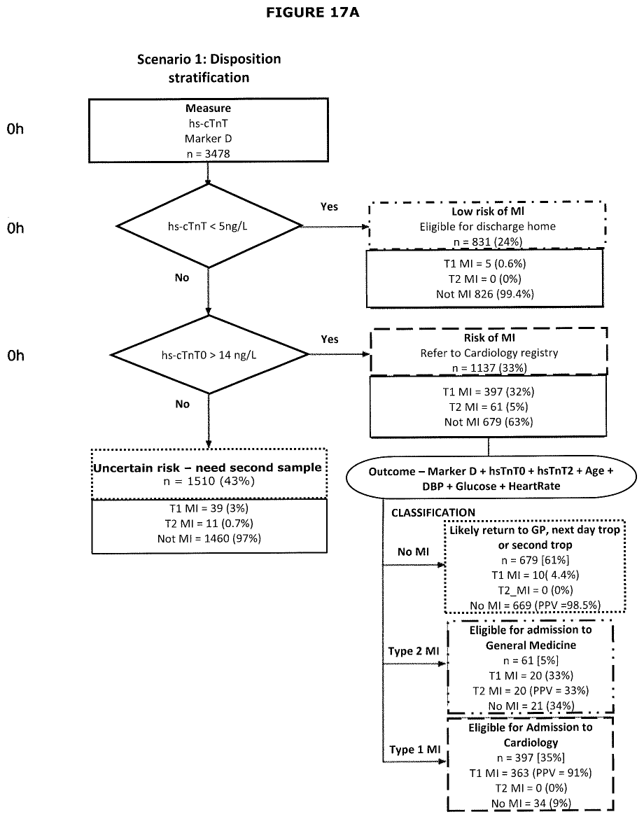

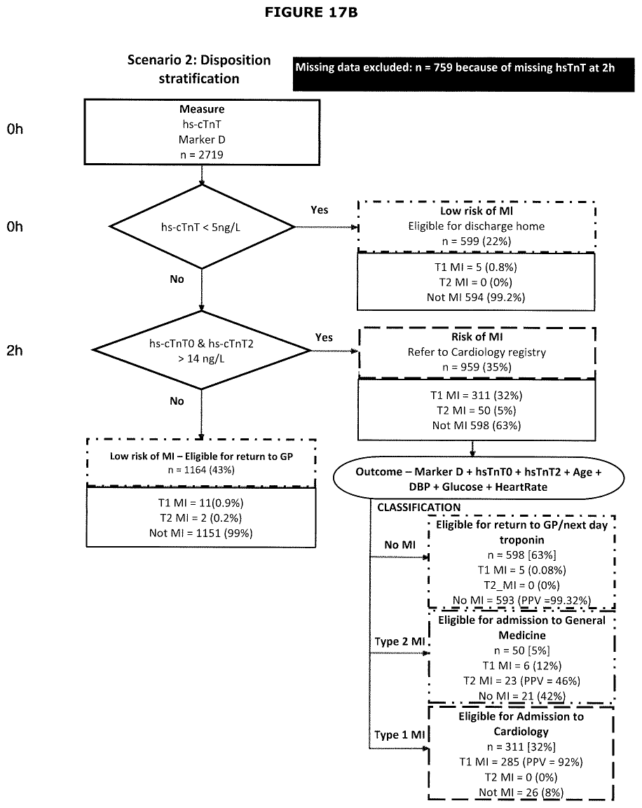

The data presented in FIG. 16, read in conjunction with Tables 22-24 below, demonstrate that a change in bTnI-IgG (i.e. “delta bTnI-IgG” or “ΔbTnI-IgG”) across a 30 minute to 10 hour window, and preferably a window of two hours, from index presentation may be used to correctly identify patients with Type 2 myocardial infarction where hsTnT is >14 ng/L at index presentation.

TABLE 22
Test Variables in the Equation - Unvariable Prediction

| | | | | | | | | 95% C.I for | |
|---|---|---|---|---|---|---|---|---|---|
| | | | | | | | | EXP(B) | |
| | | B | S.E. | Wald | df | Sig. | Exp(B) | Lower | Upper |
| Step 1^{a} | Zscore(bTnI-IgG) | −0.482 | 0.300 | 2.576 | 1 | <0.108 | 0.618 | 0.343 | 1.112 |
| | Zscore(ΔbTnI-IgG) | 0.699 | 0.219 | 10.147 | 1 | 0.001 | 2.012 | 1.309 | 3.094 |
| | Constant | −2.448 | 0.258 | 92.952 | 1 | <0.001 | 0.083 | | |

TABLE 23
Test Variables in the Equation - Multivariable Prediction

| | | | | | | | | 95% C.I for | |
|---|---|---|---|---|---|---|---|---|---|
| | | | | | | | | EXP(B) | |
| | | B | S.E. | Wald | df | Sig. | Exp(B) | Lower | Upper |
| Step 1^{a} | Zscore(bTnI-IgG) | −0.634 | 0.323 | 3.853 | 1 | 0.050 | 0.530 | 0.282 | 0.999 |
| | Zscore(ΔbTnI-IgG) | 0.685 | 0.241 | 8.058 | 1 | 0.005 | 1.984 | 1.236 | 3.185 |
| | Zscore(heart rate) | 0.620 | 0.131 | 22.363 | 1 | <0.001 | 1.859 | 1.438 | 2.404 |
| | Zscore(DPB) | −0.439 | 0.199 | 4.856 | 1 | 0.028 | 0.645 | 0.436 | 0.953 |
| | Zscore(hemaglobin) | 0.401 | 0.228 | 3.102 | 1 | 0.078 | 1.493 | 0.956 | 2.333 |
| | Constant | −2.956 | 0.319 | 85.782 | 1 | <0.001 | 0.052 | | |

TABLE 24
ROC performance of ΔbTnI-IgG for Dx of Type 2 Myocardial Infarction

| | | | | Asymptotic 95% Confidence | |
|---|---|---|---|---|---|
| | | Std. | Asymptotic | Interval | |
| Test Result Variable(s) | Area | Error^{a} | Sig.^{b} | Lower Bound | Upper Bound |
| AbTnI-IgG_(0-2 h) | 0.646 | 0.043 | 0.001 | 0.561 | 0.732 |
| hsTnT0 | 0.488 | 0.042 | 0.782 | 0.405 | 0.571 |
| heart rate + DPB + platelet + | 0.711 | 0.050 | 0.000 | 0.612 | 0.810 |
| hemaglobin | | | | | |
| heart rate + DPB + | 0.793 | 0.035 | 0.000 | 0.725 | 0.861 |
| hemaglobin + ΔMD^{#} | | | | | |

^{#}P = 0.018

### Example 13: Utility of bTnI-IgG To Predict New Acute Coronary Syndromes

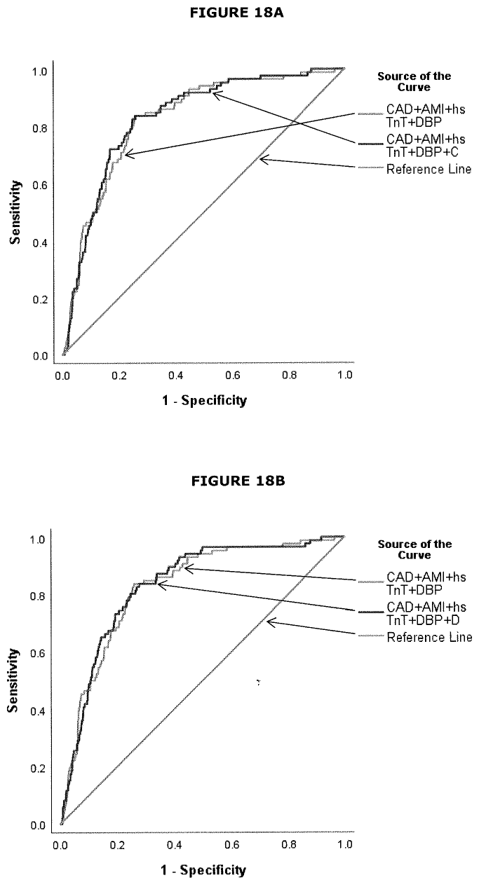

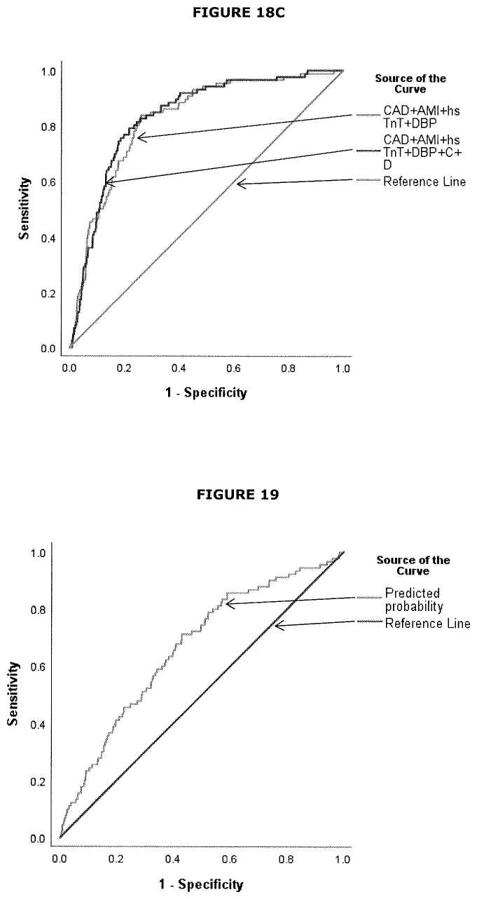

The Figures relevant to this utility are FIGS. 18-20 read in conjunction with Tables 25-31 which follow.

bTnI-IgG was shown to enhance the performance of existing clinical variables (e.g. cardiovascular disease status, acute myocardial infarction status, diastolic blood pressure, IGFBP3 etc) as indicated to predict a new acute coronary syndrome event within the next year (365 days) of presentation.

TABLE 25
Regression Analysis for Utility of bTnI-IgG to Px New ACS <365 days Non-Index MI

| | | | | | | | | 95% C.I for | |
|---|---|---|---|---|---|---|---|---|---|
| | | | | | | | | EXP(B) | |
| | | B | S.E. | Wald | df | Sig. | Exp(B) | Lower | Upper |
| Step 1^{a} | HxCAD | 1.947 | 0.377 | 26.613 | 1 | <0.001 | 7.010 | 3.345 | 14.691 |
| | Zscore(DBP) | −0.282 | 0.125 | 5.081 | 1 | 0.024 | 0.755 | 0.591 | 0.964 |
| | Log10_HsTnI0 | 0.417 | 0.196 | 4.517 | 1 | 0.034 | 1.517 | 1.033 | 2.227 |
| | HxAMI | 0.465 | 0.300 | 2.400 | 1 | 0.121 | 1.592 | 0.884 | 2.866 |
| | Zscore(IGFBP3) | −0.238 | 0.123 | 3.779 | 1 | 0.052 | 0.788 | 0.620 | 1.002 |
| | Zscore(bTnI-IgG) | −0.251 | 0.130 | 3.716 | 1 | 0.054 | 0.778 | 0.603 | 1.004 |
| | Constant | −5.353 | 0.303 | 311.548 | 1 | <0.001 | 0.005 | | |

TABLE 26
ROC Analysis for Utility of Maker Combinations to Px New ACS <365 Days Non-Index MI

| | | | | Asymptotic 95% Confidence | |
|---|---|---|---|---|---|
| | | Std. | Asymptotic | Interval | |
| Test Result Variable(s) | Area | Error^{a} | Sig.^{b} | Lower Bound | Upper Bound |
| CAD + AMI + hsTnT + DBP | 0.826 | 0.021 | 0.000 | 0.785 | 0.866 |
| CAD + AMI + hsTnT + DBP + IGFBP3 | 0.830 | 0.020 | 0.000 | 0.791 | 0.870 |

TABLE 27
ROC Analysis for Utility of Maker Combinations to Px New ACS <365 Days Non-Index MI

| | | | | Asymptotic 95% Confidence | |
|---|---|---|---|---|---|
| | | Std. | Asymptotic | Interval | |
| Test Result Variable(s) | Area | Error^{a} | Sig.^{b} | Lower Bound | Upper Bound |
| CAD + AMI + hsTnT + DBP | 0.826 | 0.021 | 0.000 | 0.786 | 0.867 |
| CAD + AMI + hsTnT + DBP + bTnI-IgG | 0.832 | 0.020 | 0.000 | 0.793 | 0.872 |

TABLE 28
ROC Analysis for Utility of Maker Combinations to Px New ACS <365 Days Non-Index MI

| | | | | Asymptotic 95% Confidence | |
|---|---|---|---|---|---|
| | | Std. | Asymptotic | Interval | |
| Test Result Variable(s) | Area | Error^{a} | Sig.^{b} | Lower Bound | Upper Bound |
| CAD + AMI + hsTnT + DBP | 0.826 | 0.021 | 0.000 | 0.785 | 0.866 |
| CAD + AMI + hsTnT + DBP + bTnI- | 0.835 | 0.020 | 0.000 | 0.797 | 0.874 |
| IgG + IGFBP3 | | | | | |

Further analysis as to the utility of bTnI-IgG was interrogated in a patient cohort who were either of T2 myocardial infarction, unstable angina pectoris or >70% artery occlusion (measured by imaging) to then predict new ACS<365 days. The data is presented in FIG. 19 and Tables 29-30 below.

TABLE 29
Test Variables in the Equation - Unvariable Prediction

| | | | | | | | | 95% C.I for | |
|---|---|---|---|---|---|---|---|---|---|
| | | | | | | | | EXP(B) | |
| | | B | S.E. | Wald | df | Sig. | Exp(B) | Lower | Upper |
| Step 1^{a} | Zscore(bTnI-IgG) | −0.293 | 0.133 | 4.844 | 1 | 0.028 | 0.746 | 0.575 | 0.968 |
| | Zscore(IGFBP3) | −0.273 | 0.116 | 5.498 | 1 | 0.019 | 0.761 | 0.606 | 0.956 |
| | Constant | −2.286 | 0.122 | 349.706 | 1 | <0.001 | 0.102 | | |

TABLE 30
Test Variables in the Equation - Multivariable Prediction

| | | | | | | | | 95% C.I for | |
|---|---|---|---|---|---|---|---|---|---|
| | | | | | | | | EXP(B) | |
| | | B | S.E. | Wald | df | Sig. | Exp(B) | Lower | Upper |
| Step 1^{a} | SexMale | −0.518 | 0.249 | 4.336 | 1 | 0.037 | 0.596 | 0.366 | 0.970 |
| | RiskChol | −0.499 | 0.258 | 3.736 | 1 | 0.053 | 0.607 | 0.366 | 1.007 |
| | HxAMI | 0.648 | 0.244 | 7.067 | 1 | 0.008 | 1.912 | 1.186 | 3.083 |
| | RiskDM | 0.474 | 0.246 | 3.701 | 1 | 0.054 | 1.606 | 0.991 | 2.601 |
| | Zscore(bTnI-IgG) | −0.242 | 0.135 | 3.201 | 1 | 0.074 | 0.785 | 0.63 | 1.023 |
| | Zscore(IGFBP3) | −0.252 | 0.121 | 4.352 | 1 | 0.037 | 0.777 | 0.613 | 0.985 |
| | Constant | −2.000 | 0.269 | 55.402 | 1 | <0.001 | 0.135 | | |

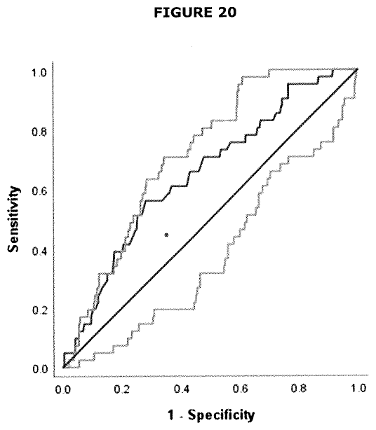

Finally, the utility of bTnI-IgG was interrogated in a patient cohort who either had a positive angiogram, a positive stress test or unstable angina pectoris, who also had no index MI, for its ability to predict a new ACS<365 days. These data are presented in FIG. 20 and Tables 31-32 as follows.

TABLE 31
Test Variables in the Equation - Full Model

| | | | | | | | | 95% C.I for | |
|---|---|---|---|---|---|---|---|---|---|
| | | | | | | | | EXP(B) | |
| | | B | S.E. | Wald | df | Sig. | Exp(B) | Lower | Upper |
| Step 1^{a} | HxCAD | 0.920 | 0.441 | 4.348 | 1 | 0.037 | 2.508 | 1.057 | 5.953 |
| | ZScore(hemaglobin) | −0.458 | 0.221 | 4.302 | 1 | 0.038 | 0.633 | 0.410 | 0.975 |
| | RiskChol | −0.769 | 0.448 | 2.940 | 1 | 0.086 | 0.464 | 0.193 | 1.116 |
| | Zscore(bTnI-IgG) | −0.632 | 0.239 | 7.002 | 1 | 0.008 | 0.531 | 0.333 | 0.849 |
| | Constant | −2.180 | 0.463 | 22.154 | 1 | <0.001 | 0.113 | | |

TABLE 32
ROC Performance of bTnI-IgG to Px ACS <365days

| | AUC | Sens. | Spec. | NPV | PPV | P value |
|---|---|---|---|---|---|---|
| Chol + Hb + | 0.650 | 95 | 23 | 97.0 | 14.8 | |
| CAD | | | | | | |
| Chol + Hb + | 0.718 | 95 | 40 | 98.3 | 18.2 | 0.051 |
| CAD + | | | | | | |
| bTnI-IgG | | | | | | |
| bTnI-IgG | 0.380 | | | | | |

### Example 14: Utility of bTnI-IgG To Predict Positive Stress Test

The utility of bTnI-IgG to predict a positive stress test in a dedicated stress test cohort was next examined.

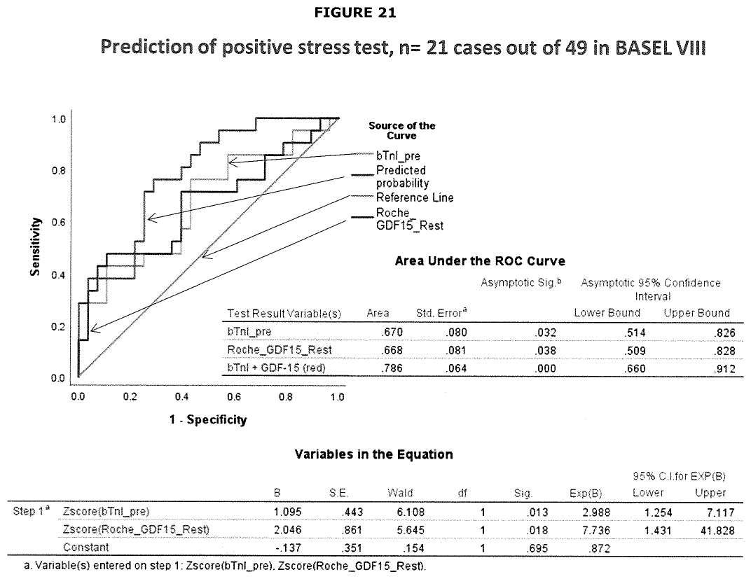

These data are presented in FIG. 21 and demonstrate that bTnI-IgG could be used alongside GDF15 and/or troponin to predict a positive stress test outcome in a stress test cohort (21/49; BASEL VIII). The data which accompanies FIG. 21 is presented below in Tables 33 and 34.

TABLE 33
Test Variables in the Equation - Full Model

| | | | | | | | | 95% C.I for | |
|---|---|---|---|---|---|---|---|---|---|
| | | | | | | | | EXP(B) | |
| | | B | S.E. | Wald | df | Sig. | Exp(B) | Lower | Upper |
| Step 1^{a} | Zscore (bTnI-IgG_pre test) | 1.095 | 0.443 | 6.108 | 1 | 0.013 | 2.988 | 1.254 | 7.117 |
| | ZScore(Roches_GDF15_rest) | 2.046 | 0.861 | 5.645 | 1 | 0.018 | 7.736 | 1.431 | 41.828 |
| | Constant | −0.137 | 0.351 | 0.154 | 1 | 0.695 | 0.872 | | |

TABLE 34
ROC Performance of bTnI-IgG to Px Positive Stress Test

| | | | | Asymptotic 95% Confidence | |
|---|---|---|---|---|---|
| | | Std. | Asymptotic | Interval | |
| Test Result Variable(s) | Area | Error^{a} | Sig.^{b} | Lower Bound | Upper Bound |
| bTnI-IgG_pre stress test | 0.670 | 0.080 | 0.032 | 0.514 | 0.826 |
| Roche_GDF15_rest | 0.668 | 0.081 | 0.038 | 0.509 | 0.828 |
| bTnI-IgG + GDF15 | 0.786 | 0.064 | 0.000 | 0.660 | 0.912 |

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

### REFERENCES

1. Munro A R, Jerram T, Morton T, Hamilton S, 2015. Use of an accelerated diagnostic pathway allows rapid and safe discharge of 70% of chest pain patients from the emergency department. NZ Med J 128:62-71.
2. Ministry of Health, 2012. Mortality and demographic data 2009. Wellington: New Zealand.
3. Thygesen K, Alpert J S, Jaffe A S, Simoons M L, Chaitman B R, White H D; Joint ESC/ACCF/AHA/WHFTask Force for the Universal Definition of Myocardial Infarction, 2012. Third Universal Definition of Myocardial Infarction. Circulation 126:2020-2035.
4. eichlin T, Hochholzer W, Bassetti S, Steuer S, Stelzig C, Hartwiger S, Biedert S, Schaub N, Buerge C, Potocki M, Noveanu M, Breidthardt T, Twerenbold R, Winkler K, Bingisser R, Mueller C, 2009. Early diagnosis of myocardial infarction with sensitive cardiac troponin assays. New England J. Med. 361:858-867.
5. Keller T, Zeller T, Peetz D, Tzikas S, Roth A, Czyz E, Bickel C, Baldus S, Warnholtz A, Fröhlich M, Sinning C R, Lackner K J, Munzel T F, Blankenberg S, 2009. Sensitive troponin I assay in early diagnosis of myocardial infarction. New England J. Med. 361:868-877.
6. Mueller C, 2014. Biomarkers and acute coronary syndromes: an update. Eur. Heart J. 35:552-556.
7. Deckers J W, 2013. Classification of myocardial infarction and unstable angina: a re-assessment. Int. J. Cardiol. 167:2387-2390.
8. Thygesen K, Alpert J S, Jaffe A S, et al. Fourth Universal Definition of Myocardial Infarction (2018). J Am Coll Cardiology 72(18):2231-2264.
9. Hanley J A, McNeil B J. A method of comparing the areas under receiver operating characteristic curves derived from the same cases. Radiology (1983) 148(3):839-43.

1. A method for enhancing the diagnostic performance of a cardiac troponin T (cTnT) assay for the diagnosis of an acute myocardial infarction in a patient, the method comprising:
(i) determining the level of a bound protein complex comprising cardiac Troponin I (cTnI) and a human immunoglobulin G (IgG) (bTnI-IgG) in a biological sample obtained from the patient which comprises:
(a) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
(b) washing the reaction mix from (a) to remove non-selectively bound analytes;
(c) contacting the reaction mix from (b) with an anti-IgG detection antibody, for a time and under conditions sufficient for the anti-IgG detection antibody to selectively bind to the bTnI-IgG complex; and
(d) determining the level of bTnI-IgG in the biological sample;
(ii) comparing the level of bTnI-IgG from (i) against a reference standard from a control population
wherein, when the level of bTnI-IgG in the biological sample is lower relative to the reference standard from a control population it is combined with cTnT to improve the diagnostic accuracy of cTnT which is achieved in the absence of bTnI-IgG.

2. The method according to claim 1, wherein the improved diagnostic accuracy of the cTnT assay comprises improved assay specificity.

3. The method according to claim 1 or claim 2 wherein the cTnT is high sensitivity cardiac troponin T assay (hsTnT).

4. The method according to any one of claims 1 to 3 wherein the additional clinical variables to patient sex and electrocardiogram status are considered.

5. A method for diagnosing an acute coronary syndrome in a patient, the method comprising:
(i) determining the level of a bound protein complex comprising cardiac Troponin I (cTnI) and an immunoglobulin G (IgG) (bTnI-IgG) in a biological sample obtained from the patient which comprises:
(a) contacting the biological sample with a reaction mix comprising an anti-cTnI capture antibody which selectively binds to an equivalent region comprising amino acids 80-97 of cTnI as defined by SEQ ID NO: 1, for a time and under conditions sufficient for the anti-cTnI capture antibody to selectively bind to the bTnI-IgG complex;
(b) washing the reaction mix from (a) to remove non-selectively bound analytes;
(c) contacting the reaction mix from (b) with an anti-IgG detection antibody, for a time and under conditions sufficient for the anti-IgG detection antibody to selectively bind to the bTnI-IgG complex; and
(d) determining the level of bTnI-IgG in the biological sample;
(ii) comparing the level of bTnI-IgG from (i) against a reference standard from a control population
wherein, a lower level of bTnI-IgG in the biological sample measured relative to the reference standard from a control population is indicative that the patient has an acute coronary syndrome.

6. The method according to claim 5, wherein the acute coronary syndrome is an acute myocardial infarction or is unstable angina pectoris.

7. The method according to any one of claims 1 to 6, wherein the measured level of bTnI-IgG is less than or equal to 15 ng/L.

8. The method according to any one of claims 1 to 7 wherein the anti-cTnI antibody selectively binds to SEQ ID NO: 1.

9. The method according to any one of claims 1 to 8, wherein the anti-cTnI antibody is a monoclonal antibody.

10. The method according to any one of claims 1 to 9, wherein the anti-cTnI monoclonal antibody is produced by Jackson Labs, Abcam, ThermoFisher.

11. The method according to any one of claims 1 to 10, wherein the anti-cTnI antibody is immobilized on a solid substrate.

12. The method according to any one of claims 1 to 11, wherein the anti-IgG antibody is a human monoclonal antibody.

13. The method according to any one of claims 1 to 12 wherein the anti-IgG antibody comprises a detectable label.

14. The method according to any one of claims 1 to 13, wherein the detectable label is an enzymatic detection label.
